(19) United States
(12) Patent Application Publication
(10) Pub. No.: US 2025/0121110 A1
(43) Pub. Date: Apr. 17, 2025

(54) Sterilization Indicator Reading Apparatus and Method

(71) Applicant: Solventum Intellectual Properties Company, Maplewood, MN (US)

(72) Inventors: Peter D. Ludowise, Cottage Grove, MN (US); Jeffrey M. Flynn, Osceola, WI (US)

(21) Appl. No.: 18/695,948

(22) PCT Filed: Sep. 8, 2022
(86) PCT No.: PCT/IB2022/058471

§ 371 Date: Mar. 27, 2024

Related U.S. Application Data
(60) Provisional application No. 63/261,694, filed on Sep. 27, 2021.

(51) Int. Cl. A61L 2/28

(52) U.S. Cl. CPC A61L 2/28; A61L 2202/14; A61L 2202/24

(57) ABSTRACT

A sterilization indicator reading apparatus includes a housing, at least one well formed from a portion of the housing and dimensioned to receive a sterilization indicator, at least one heating element thermally coupled to a coupling portion of the well, at least one sensor configured to sense at least one parameter associated with the sterilization indicator received within the well and generate response signals upon sensing the parameter, and a processor communicably coupled to the heating element and the sensor. The processor is configured to control the heating element to achieve a first preset temperature of the well from a first instance of time after receiving a first response signal from the sensor, and to control the heating element to achieve a second preset temperature of the well upon receiving a second response signal from the sensor, or after a predetermined time duration from the first instance of time.

### TECHNICAL FIELD

The present disclosure generally relates to a sterilization indicator reading apparatus, and in particular to a sterilization indicator reading apparatus that facilitates rapid reading of sterilization indicators, and a method of controlling the sterilization indicator reading apparatus.

### BACKGROUND

In a variety of industries, such as the health care industry, but also in other industrial applications, it may be necessary to monitor effectiveness of processes used to sterilize equipment such as medical devices, instruments, and other disposable and non-disposable articles. A sterilization cycle is generally defined as a process of completely destroying all viable sources of biological activity, such as microorganisms, including structures such as viruses and spores. As a standard practice, hospitals or other institutions include a sterilization indicator with a batch of articles to assay the lethality of the sterilization cycle/process. Both biological and chemical sterilization indicators may be used.

The biological sterilization indicator may include a known quantity of test microorganisms, for example. Geobacillus stearothermophilus (formerly Bacillus stearothermophilus) or Bacillus atrophacus (formerly Bacillus subtilis) spores, which may be many times more resistant to particular sterilization processes than other contaminating organisms. After the exposure of the indicator to the sterilization process, the sources of biological activity (e.g., spores) may be incubated in a liquid nutrient medium to determine an effectiveness of the sterilization process, for example, whether any of the sources survived the sterilization process, with source metabolism and/or growth indicating that the sterilization process may be insufficient to destroy all the sources of biological activity.

Reading apparatuses may be used to read the biological sterilization indicators after the biological sterilization indicators undergo the sterilization process to determine effectiveness of the sterilization process, i.e., whether the sterilization process was able to effectively destroy the test microorganisms of the biological sterilization indicators. However, conventional reading apparatuses may be slow to read the biological sterilization indicators, thereby causing a delay in obtaining a result indicative of the effectiveness of the sterilization process.

### SUMMARY

In a first aspect, the present disclosure provides a sterilization indicator reading apparatus. The sterilization indicator reading apparatus includes a housing including a top portion, and a bottom portion opposite the top portion. The sterilization indicator reading apparatus further includes at least one well formed from a portion of the housing, accessible from the top portion, and oriented along a well axis from the top portion to the bottom portion. The at least one well is dimensioned to receive at least a portion of a sterilization indicator having spores and a substance fluorescently responsive to a spore viability. The spores are responsive to an environmental condition in a sterilizer. The sterilization indicator reading apparatus further includes at least one heating element thermally coupled to a coupling portion of the at least one well. The sterilization indicator reading apparatus further includes at least one sensor configured to sense at least one parameter associated with the sterilization indicator received at least partially within the at least one well and generate one or more response signals upon sensing the at least one parameter. The sterilization indicator reading apparatus further includes a processor communicably coupled to the at least one heating element and the at least one sensor. The processor is configured to control the at least one heating element to achieve a first preset temperature of at least the coupling portion of the at least one well from a first instance of time after receiving a first response signal from the at least one sensor. The processor is further configured to control the at least one heating element to achieve a second preset temperature of at least the coupling portion of the at least one well upon receiving a second response signal from the at least one sensor, or after a predetermined time duration from the first instance of time. The second preset temperature is different from the first preset temperature.

In a second aspect, the present disclosure provides a sterilization indicator reading apparatus. The sterilization indicator reading apparatus includes a housing including a top portion, and a bottom portion opposite the top portion. The sterilization indicator reading apparatus further includes at least one well formed from a portion of the housing, accessible from the top portion, and oriented along a well axis from the top portion to the bottom portion. The at least one well is dimensioned to receive at least a portion of a sterilization indicator having spores and a substance fluorescently responsive to a spore viability. The spores are responsive to an environmental condition in a sterilizer. The at least one well includes a heater block. The sterilization indicator reading apparatus further includes at least one heating element thermally coupled to the heater block of the at least one well. The sterilization indicator reading apparatus further includes at least one sensor configured to sense at least one parameter associated with the sterilization indicator received at least partially within the at least one well and generate one or more response signals upon sensing the at least one parameter. The sterilization indicator reading apparatus further includes a processor communicably coupled to the at least one heating element and the at least one sensor. The processor is configured to control the at least one heating element in order to achieve a temperature setpoint of the heater block. The processor is further configured to set the temperature setpoint of the heater block of the at least one well to a first preset temperature from a first instance of time after receiving a first response signal from the at least one sensor. The processor is further configured to set the temperature setpoint of the heater block of the at least one well to a second preset temperature upon receiving a second response signal from the at least one sensor, or after a predetermined time duration from the first instance of time. The processor is further configured to set the temperature setpoint of the heater block of the at least one well to a third preset temperature prior to receiving the first response signal. Each of the second preset temperature and the third preset temperature is different from the first preset temperature.

In a third aspect, the present disclosure provides a method of controlling a sterilization indicator reading apparatus. The sterilization indicator reading apparatus has a housing, at least one well formed from a portion of the housing, at least one heating element thermally coupled to a coupling portion of the at least one well, at least one sensor, and a processor communicably coupled to the at least one heating element and the at least one sensor. The method includes receiving, via the processor, one or more response signals from the at least one sensor indicative of at least one parameter associated with a sterilization indicator received at least partially within the at least one well. The sterilization indicator has spores and a substance fluorescently responsive to a spore viability. The spores are responsive to an environmental condition in a sterilizer. The method further includes controlling, via the processor, the at least one heating element to achieve a first preset temperature of at least the coupling portion of the at least one well from a first instance of time after receiving a first response signal from the at least one sensor. The method further includes controlling, via the processor, the at least one heating element to achieve a second preset temperature of at least the coupling portion of the at least one well upon receiving a second response signal from the at least one sensor, or after a predetermined time duration from the first instance of time. The second preset temperature is different from the first preset temperature.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic front perspective view of a sterilization indicator;

FIG. 2 is a schematic front perspective view of a system including a sterilization indicator reading apparatus with the sterilization indicator received therein according to an embodiment of the present disclosure;

FIG. 3 is a schematic cross-sectional view of the sterilization indicator reading apparatus taken along a line 1-1 of FIG. 2;

FIG. 4A is a schematic perspective view of a heater block of the sterilization indicator reading apparatus according to an embodiment of the present disclosure;

FIG. 4B is a schematic cross-sectional perspective view of the heater block taken along a line 2-2 of FIG. 4A;

FIG. 4C is a schematic cross-sectional perspective view of the heater block taken along a line 3-3 of FIG. 4A;

FIG. 5A is a schematic front view of the sterilization indicator reading apparatus including a heater block assembly with some elements of the sterilization indicator reading apparatus not shown according to an embodiment of the present disclosure;

FIG. 5B is a schematic rear view of the sterilization indicator reading of FIG. 5A;

FIG. 6 is a schematic block diagram of the sterilization indicator reading apparatus according to an embodiment of the present disclosure;

FIG. 7 is a graph depicting a variation of a desired temperature of a coupling portion of the sterilization indicator reading apparatus with respect to time according to an embodiment of the present disclosure;

FIG. 8 is a graph depicting a variation of the temperature setpoint of the coupling portion of the sterilization indicator reading apparatus according to another embodiment of the present disclosure;

FIG. 9 is a flowchart depicting various steps of a method of controlling a sterilization indicator reading apparatus according to an embodiment of the present disclosure;

FIG. 10 is a graph depicting a variation of temperature of a heater block with respect to time and a variation of temperature of a substance of a sterilization indicator due to the variation of temperature of the heater block with respect to time;

FIG. 11 is a graph depicting a variation of temperature of the substance of the sterilization indicator incubated in the sterilization indicator reading apparatus of the present disclosure with respect to time and a variation of temperature of the substance of the sterilization indicator incubated in a conventional reading apparatus with respect to time;

FIG. 12 is a graph depicting a variation of temperatures of heater blocks controlled differently with respect to time and a variation of respective temperatures of sterilization indicators due to the variation of temperatures of the heater blocks with respect to time;

FIG. 13 is a graph depicting a variation of fluorescence signals of sterilization indicators incubated in the sterilization indicator reading apparatus at different temperatures with respect to time;

FIG. 14 is a graph depicting a variation of average fluorescence signals of sterilization indicators incubated in the sterilization indicator reading apparatus at different temperatures with respect to time; and

FIG. 15 is a graph depicting a variation of a fluorescence signal of a sterilization indicator incubated in the sterilization indicator reading apparatus with respect to time.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein. “at least one of A and B” should be understood to mean “only A, only B, or both A and B”.

As used herein, the term “sterilization” generally refers to a process of eliminating all bacteria and other living organisms from the surfaces of instruments, medical devices, implants, and other articles used in sterile surgical procedures. A conventional thermal sterilization process uses steam under pressure. Low-temperature chemical sterilization processes use ethylene oxide, hydrogen peroxide, hydrogen peroxide/plasma, or peracetic acid in liquid or vapor form as the sterilant, as well as gamma irradiation and electron beam sterilization.

As used herein, the term “sterilizer” refers to a system or an apparatus that can carry out a sterilization cycle, i.e., a process of completely destroying all viable sources of biological activity, such as microorganisms, including structures such as viruses and spores.

As used herein, the term “result” refers to an outcome indicative of an effectiveness of a sterilization cycle that can be determined by a sterilization indicator reading apparatus by reading a sterilization indicator that has undergone the sterilization cycle. The result may be positive or negative. A positive result refers to an unsuccessful sterilization of the sterilization indicator after undergoing the sterilization process. The positive result may be determined by the sterilization indicator reading apparatus upon detection of a presence of biological activity (for example, by detection of unsterilized microorganisms) in the sterilization indicator after undergoing the sterilization process. A negative result refers to a successful sterilization of the sterilization indicator after undergoing the sterilization process. The negative result may be determined by the sterilization indicator reading apparatus when the presence of biological activity is not detected in the sterilization indicator after undergoing the sterilization process. The negative result is indicative of effective sterilization. In other words, the negative result indicates that microorganisms in the sterilization indicator are effectively killed during the sterilization process.

Various sterilization indicators may be used for various different sterilization processes using steam, hydrogen peroxide gas, ethylene oxide, and the like. The sterilization indicators may carry a biological agent. The sterilization indicators are typically placed in a test package within a load containing articles to be sterilized. The sterilization indicators may indicate successful sterilization when the biological agent has been killed. The biological agent carried by the sterilization indicators is typically a test organism which is many times more resistant to the sterilization process than most organisms that are present due to natural contamination. The biological agent may include microorganisms, such as endospores, bacterial spores, or the like.

Reading apparatuses may be used to read the biological sterilization indicators after the biological sterilization indicators undergo the sterilization process to determine effectiveness of the sterilization process, i.e., whether the sterilization process was able to effectively destroy the test microorganisms of the biological sterilization indicators. However, conventional reading apparatuses may be slow to read the biological sterilization indicators, thereby causing a delay in obtaining a result indicative of the effectiveness of the sterilization process.

The present disclosure provides a sterilization indicator reading apparatus. The sterilization indicator reading apparatus includes a housing including a top portion, and a bottom portion opposite the top portion. The sterilization indicator reading apparatus further includes at least one well formed from a portion of the housing, accessible from the top portion, and oriented along a well axis from the top portion to the bottom portion. The at least one well is dimensioned to receive at least a portion of a sterilization indicator having spores and a substance fluorescently responsive to a spore viability. The spores are responsive to an environmental condition in a sterilizer. The sterilization indicator reading apparatus further includes at least one heating element thermally coupled to a coupling portion of the at least one well. The sterilization indicator reading apparatus further includes at least one sensor configured to sense at least one parameter associated with the sterilization indicator received at least partially within the at least one well and generate one or more response signals upon sensing the at least one parameter. The sterilization indicator reading apparatus further includes a processor communicably coupled to the at least one heating element and the at least one sensor. The processor is configured to control the at least one heating element to achieve a first preset temperature of at least the coupling portion of the at least one well from a first instance of time after receiving a first response signal from the at least one sensor. The processor is further configured to control the at least one heating element to achieve a second preset temperature of at least the coupling portion of the at least one well upon receiving a second response signal from the at least one sensor, or after a predetermined time duration from the first instance of time. The second preset temperature is different from the first preset temperature.

The sterilization indicator reading apparatus of the present disclosure may be used to rapidly and accurately read the sterilization indicators that have undergone a sterilization process to determine an efficacy/effectiveness of the sterilization process. In other words, the sterilization indicator reading apparatus of the present disclosure may reduce a time taken to obtain a result (also known as TTR (time to result)) indicative of the effectiveness of the sterilization process.

The sterilization indicator reading apparatus may receive at least a portion of the sterilization indicator in the at least one well. The at least one sensor may sense the at least one parameter associated with the sterilization indicator and generate the one or more response signals upon sensing the at least one parameter.

The processor may automatically adjust the temperature of the sterilization indicator from the first preset temperature to the second preset temperature. Specifically, the processor may control the at least one heating element to achieve the first preset temperature of at least the coupling portion from the first instance of time after receiving the first response signal. The first preset temperature may speed up and facilitate a first phase of incubation of the spores of the sterilization indicator (e.g., an α-glucosidase reaction phase). Subsequently, the processor may control the at least one heating element to achieve the second preset temperature of at least the coupling portion upon receiving the second response signal from the at least one sensor, or after a predetermined time duration from the first instance of time. The second preset temperature may speed up and facilitate a second phase of incubation of the spores of the sterilization indicator (e.g., a growth phase). Therefore, the sterilization indicator reading apparatus may effectively control the temperature of the coupling portion to reduce the time taken to read the sterilization indicator and obtain the result indicative of the effectiveness of the sterilization process.

In some examples, the at least one well of the sterilization indicator reading apparatus may include a plurality of wells thermally isolated from each other. Therefore, each of the plurality of wells may be independently controlled. As a result, the sterilization indicator reading apparatus may simultaneously read a plurality of sterilization indicators. Moreover, the plurality of wells may be independently controlled such that the sterilization indicator reading apparatus may simultaneously read different types of sterilization indicators having different incubation requirements. Therefore, the sterilization indicator reading apparatus may effectively control the temperature of respective coupling portions of the plurality of wells to reduce the time taken to read the plurality of sterilization indicators (that may have different incubation requirements) and obtain results indicative of the effectiveness of the sterilization processes.

In some examples, a heater block may form the coupling portion of the at least one well and the at least one heating element may be thermally coupled to the heater block. The heater block may distribute heat from the heating element to the sterilization indicator. The processor may control the at least one heating element to maintain a temperature of the heater block at the first preset temperature for the predetermined time duration and control the at least one heating element to change the temperature of the heater block to the second preset temperature from the first preset temperature after the predetermined time duration. In some examples, the processor may set a temperature setpoint of the heater block to the first preset temperature for the predetermined time duration, set the temperature setpoint of the heater block to the second preset temperature after the predetermined time duration, and control the at least one heating element in order to achieve the temperature setpoint of the heater block.

In some cases, the processor may control the at least one heating element based on one or more signals received from at least one temperature sensor indicative of the temperature of the heater block. Therefore, the sterilization indicator reading apparatus may control the temperature of the heater block in a closed-loop heating system.

In one example, the processor may control the at least one heating element to achieve a third preset temperature of at least the coupling portion prior to receiving the first response signal. The third preset temperature may be different from the first preset temperature. In some cases, the third preset temperature may be greater than the first preset temperature. In such cases, the coupling portion may be preheated to the third preset temperature. Preheating the coupling portion to the third preset temperature may increase heat flow to the sterilization indicator to speed up and facilitate the first phase of incubation of the spores of the sterilization indicator. Consequently, the sterilization indicator reading apparatus may further reduce the time taken to read the sterilization indicator and to obtain the result indicative of the effectiveness of the sterilization process.

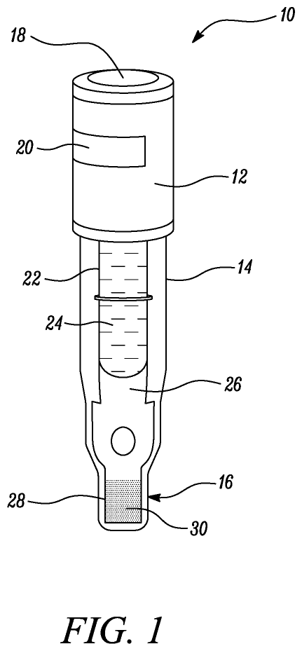

Referring now to Figures, FIG. 1 illustrates a schematic front perspective view of a sterilization indicator 10. The sterilization indicator 10 is preferably a biological sterilization indicator (e.g., a self-contained biological sterilization indicator). Examples of the biological sterilization indicator are known and are manufactured by companies such as 3M under the trade designation ATTEST, Steris (Mentor, OH) under the trade designation Verify, and Terragene (Argentina).

The sterilization indicator 10 illustrated in FIG. 1 includes a cap 12, an outer vial 14, and a growth chamber 16. The sterilization indicator 10 illustrated in FIG. 1 further includes a process indicator label 18 and an information label 20 disposed on (e.g., adhered to) the cap 12.

The process indicator label 18 may indicate whether the sterilization indicator 10 has been exposed to a sterilization process. For example, the process indicator label 18 may be configured to undergo a color change when exposed to the sterilization process. Therefore, the exposure of the sterilization indicator 10 to the sterilization process may be confirmed by observing the color change in the process indicator label 18. In some examples, the color change of the process indicator label 18 from light pink to brown may indicate that the sterilization indicator 10 has been exposed to the sterilization process.

The information label 20 may include information related to the sterilization indicator 10, such as a vial number, an experiment number, a sterilization process name, or any other information.

In the illustrated example of FIG. 1, the sterilization indicator 10 further includes a media ampoule 22 disposed within the outer vial 14 and having a substance 24, and a spore carrier 28 disposed within the growth chamber 16 and having spores 30. In other words, in the illustrated example of FIG. 1, the sterilization indicator 10 has the substance 24 and the spores 30. The substance 24 is fluorescently responsive to a spore viability (i.e., an ability of the spores 30 to survive a sterilization cycle). In other words, the substance 24 may be fluorescently responsive to a viable spore concentration (i.e., a concentration of the spores 30 that survive the sterilization cycle). Further, a reaction between the spores 30 and the substance 24 may cause the substance 24 to emit fluorescence upon absorbing electromagnetic radiation (e.g., ultraviolet light).

The spores 30 may be selected according to the sterilization process used. For example, for a steam sterilization process, Geobacillus stearothermophilus or Bacillus stearothermophilus may be used. For an ethylene oxide sterilization process, Bacillus atrophacus (formerly Bacillus subtilis) may be used. The spores 30 may be resistant to the sterilization process. The spores 30 may include, but are not limited to, Geobacillus stearothermophilus, Bacillus stearothermophilus, Bacillus subtilis, Bacillus atrophacus, Bacillus megaterium, Bacillus coagulans, Clostridium sporogenes, Bacillus pumilus, or combinations thereof.

The substance 24 (preferably liquid) may be a nutrient medium and can generally be selected to induce germination and initial outgrowth of the spores 30, if viable. The substance 24 may include one or more sugars, including, but not limited to, glucose, fructose, cellobiose, or a combination thereof. The substance 24 may also include a salt, including, but not limited to, potassium chloride, calcium chloride, or a combination thereof. In some embodiments, the substance 24 may further include an amino acid, including, but not limited to, methionine, phenylalanine, tryptophan, and the like.

In one example, the sterilization indicator 10 may include an α-glucosidase enzyme system, which is generated naturally within growing cells of Geobacillus stearothermophilus. The α-glucosidase in its active state may be detected by measuring a fluorescence produced by the enzymatic hydrolysis of the substance 24 (e.g., a non-fluorescent substrate, and 4-methylumbelliferyl-a-D-glucoside (MUG)).

Enzymes and substrates that may be suitable for use in the sterilization indicator 10 are identified in U.S. Pat. No. 5,073,488 (Matner et al.), U.S. Pat. No. 5,418,167 (Matner et al.), and U.S. Pat. No. 5,223,401 (Foltz et al.), which are incorporated herein by reference for all they disclose.

The sterilization indicator 10 may undergo a sterilization cycle/process of a sterilizer. The spores 30 are responsive to an environmental condition in the sterilizer. For example, the environmental condition in the sterilizer may partially or completely destroy biological activity of the spores 30. The environmental condition in the sterilizer may correspond to any one of the physical, gaseous, and liquid sterilization processes. In one example, the environmental condition may include a presence of pressurized steam. In another example, the environmental condition may include presence of any one of vaporized hydrogen peroxide and ethylene oxide.

The media ampoule 22 may be frangible and may be separated from the spore carrier 28. That is. the substance 24 may not be in fluid communication with the spores 30 prior to the media ampoule 22 being fractured, punctured, pierced, crushed, cracked, or the like. To allow mixing of the substance 24 with the spores 30, the outer vial 14 may further include an ampoule crusher 26 disposed between the media ampoule 22 and the spore carrier 28. The media ampoule 22 may be fractured, punctured, pierced, crushed, cracked, or the like via the ampoule crusher 26 by pressing the cap 12 towards the spore carrier 28 with a suitable force. Such a process of mixing of the substance 24 with the spores 30 may be referred to as an activation of the sterilization indicator 10. After undergoing the sterilization cycle/process of the sterilizer, the spores 30 can be exposed to the substance 24 to propagate by the activation of the sterilization indicator 10.

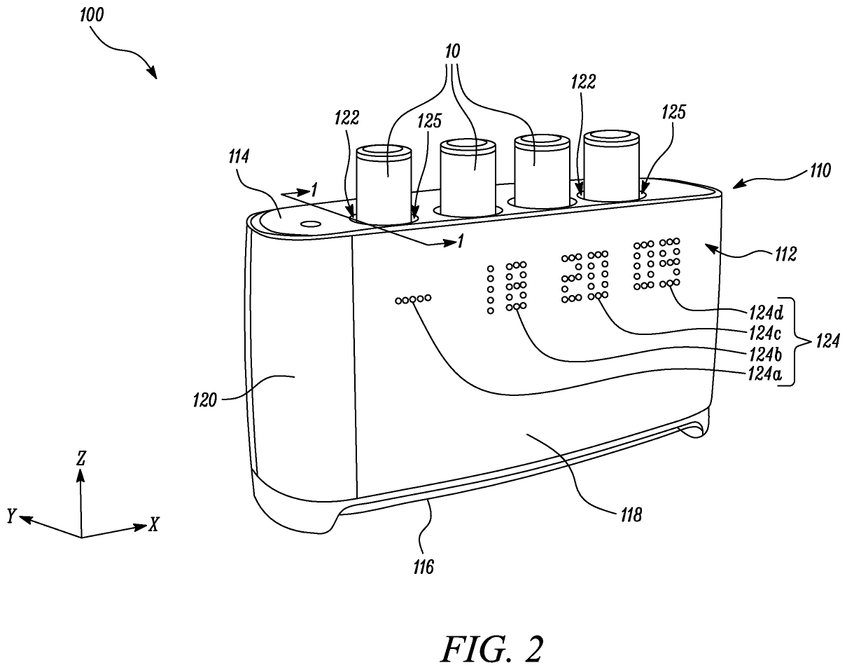

FIG. 2 illustrates a schematic front perspective view of a system 100 including the sterilization indicator 10 and a sterilization indicator reading apparatus 110 (hereinafter referred to as “the reading apparatus 110”) according to an embodiment of the present disclosure.

The reading apparatus 110 defines mutually orthogonal X, Y, and Z-axes. The X and Y-axes are in-plane axes of the reading apparatus 110, while the Z-axis is a transverse axis disposed along a height of the reading apparatus 110. In other words, the X and Y-axes are disposed along a plane of the reading apparatus 110, while the Z-axis is perpendicular to the plane of the reading apparatus 110.

The reading apparatus 110 includes a housing 112 including a top portion 114, and a bottom portion 116 opposite the top portion 114. The housing 112 may be compact, and may have an internal volume of no greater than 0.5 liter (L), no greater than 0.4 L, no greater than 0.3 L, or no greater than 0.2 L. The housing 112 is shown in FIG. 2 as being mostly rectangular, when viewed from a top to bottom direction (i.e., a direction along the Z-axis), and has a rounded rectangular or an ellipsoidal cross-section. The housing 112 may have a major side portion 118 formed along the X-axis and a minor side portion 120 formed along the Y-axis. The top portion 114 of the housing 112 may be flush with an edge of the major side portion 118 but is shown extending above the plane of the major side portion 118 in FIG. 2.

As discussed above, the housing 112 has a rounded rectangular cross-section when viewed from the top to bottom direction. In other words, the housing 112 has two straight edges and two curved edges. The two straight edges may substantially extend along the X-axis, and the two curved edges may partially extend along the Y-axis.

The reading apparatus 110 further includes at least one well 122 formed from a portion of the housing 112. The at least one well 122 is accessible from the top portion 114. In other words, the at least one well 122 can be accessed from the top portion 114 of the housing 112. The top portion 114 may include a hole 125 leading to the at least one well 122.

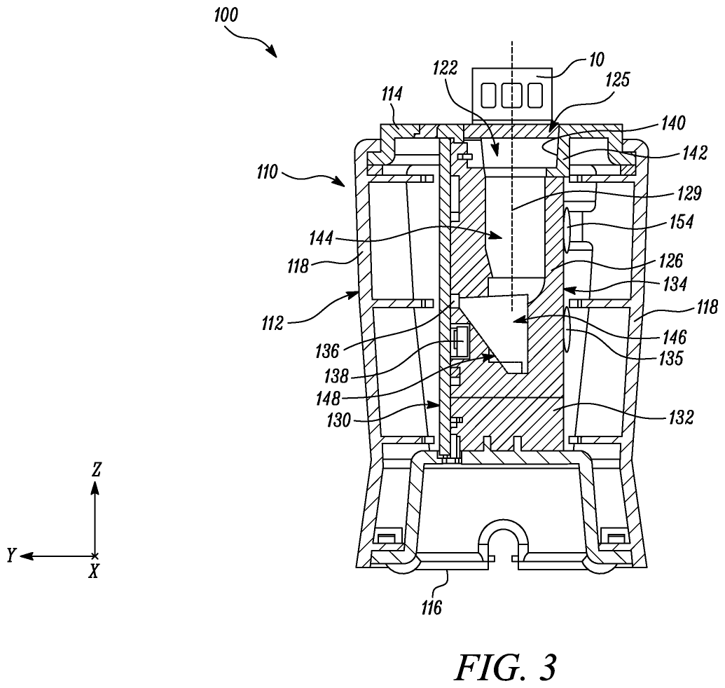

The at least one well 122 is oriented along a well axis 129 (shown in FIG. 3) from the top portion 114 to the bottom portion 116. Furthermore, the at least one well 122 is dimensioned to receive at least a portion of the sterilization indicator 10 (also shown in FIG. 1). For example, the sterilization indicator 10 may have one or more features that allow the sterilization indicator 10 to be keyed relative to the at least one well 122, such as a shelf, a protrusion, or a body shape. The at least one well 122 may receive a portion of the sterilization indicator 10 from the top portion 114 of the housing 112. In other words, a portion of the sterilization indicator 10 may be inserted into the at least one well 122 from the top portion 114 of the housing 112. In at least one embodiment, the at least one well 122 has a depth (along the Z-axis) defined by the sterilization indicator 10. As discussed above, the sterilization indicator 10 has the spores 30 (shown in FIG. 1) and the substance 24 (shown in FIG. 1) fluorescently responsive to the spore viability (i.e., the ability of the spores 30 to survive the sterilization cycle). The spores 30 are responsive to the environmental condition in the sterilizer (e.g., the environmental condition in the sterilizer may partially or completely destroy biological activity of the spores 30).

In the illustrated embodiment of FIG. 2, the at least one well 122 includes a plurality of wells 122 thermally isolated from each other (e.g., by an insulating material disposed therebetween). The plurality of wells 122 may be spaced apart from each other, and each of the plurality of wells 122 may be accessible from the top portion 114 of the housing 112. Further, in the illustrated embodiment of FIG. 2, a plurality of sterilization indicators 10 is received within corresponding wells 122 from the plurality of wells 122. In other words, the reading apparatus 110 may seat the plurality of sterilization indicators 10 within the corresponding wells 122. Specifically, in the illustrated embodiment of FIG. 2, the plurality of wells 122 includes four wells 122 arranged in a linear configuration along the X-axis. However, the plurality of wells 122 may include any number of the wells 122 arranged in any suitable configuration. The top portion 114 of the housing 112 may have a plurality of holes 125 leading to corresponding wells 122 from the plurality of wells 122.

In the illustrated embodiment of FIG. 2, the reading apparatus 110 further includes a display 124. The display 124 may visually communicate information to a user, e.g., minutes remaining, sterilization indicator pass/fail, or combinations thereof, for each well 122 from the plurality of wells 122. Each well 122 may have its own display 124 independent from another display 124 for another well 122. As shown in FIG. 2, the display 124 may include a plurality of displays 124a, 124b, 124c, 124d with one display 124 per well 122. For example, the displays 124a, 124d may be outer arrays of display elements corresponding to outer wells 122 from the plurality of wells 122 (closest to edges of the housing 112). The displays 124b, 124c may be inner arrays of the display elements corresponding to inner wells 122 from the plurality of wells 122. In at least one embodiment, a thickness of the major side portion 118 may be greater proximal to the inner wells 122 than the outer wells 122. Further, the displays 124b, 124c may be brighter than the displays 124a, 124d.

FIG. 3 illustrates a schematic cross-sectional view of the reading apparatus 110 taken along a line 1-1 of FIG. 2.

As discussed above, the at least one well 122 is oriented along the well axis 129 from the top portion 114 to the bottom portion 116. In some embodiments, the well axis 129 may be substantially parallel to the Z-axis. In other words, in some embodiments, the well 122 may be oriented substantially vertically along the Z-axis and perpendicular to a plane formed by the top portion 114 or the bottom portion 116. In some other embodiments, the well axis 129 may be inclined to the Z-axis. In other words, in some embodiments, the at least one well 122 may be askew from the Z-axis. The at least one well 122 may allow the substance 24 (shown in FIG. 1) to collect at the growth chamber 16 of the sterilization indicator 10 (preferably through gravity) when the media ampoule 22 containing the substance 24 is broken (e.g., by the ampoule crusher 26).

In the illustrated embodiment of FIG. 3, the at least one well 122 further includes a lip portion 140 that may support a portion of the sterilization indicator 10. The lip portion 140 may be formed from a portion of a wall 142 and keyed to the sterilization indicator 10. In the illustrated embodiment of FIG. 3, the at least one well 122 has a first (e.g., upper) chamber 144 and a second (e.g., lower) chamber 146. The first chamber 144 may support a larger diameter of an upper portion of the sterilization indicator 10. The second chamber 146 may accommodate a smaller diameter of a lower (i.e., tapering) portion of the sterilization indicator 10. The second chamber 146 may have a smaller perimeter than a perimeter of the first chamber 144 (measured from the smallest perimeter of the corresponding portions of the sterilization indicator 10). In some examples, the wall 142 may be configured to block ambient light from an environment from reaching the second chamber 146 of the at least one well 122.

The at least one well 122 further includes a coupling portion 134. The coupling portion 134 may be a portion of the at least one well 122 that receives at least a portion of the sterilization indicator 10. Upon receiving a portion of the sterilization indicator 10, the coupling portion 134 may be thermally coupled to the sterilization indicator 10. The coupling portion 134 may transfer heat to the sterilization indicator 10 by conduction.

The reading apparatus 110 further includes at least one heating element 135 thermally coupled to the coupling portion 134 of the at least one well 122. The at least one heating element 135 may be thermally coupled to the coupling portion 134, such that the at least one heating element 135 can heat the sterilization indicator 10.

In the illustrated embodiment of FIG. 3, the reading apparatus 110 further includes at least one heater block 126 (interchangeably referred to as “the heater block 126”) forming the coupling portion 134 of the at least one well 122. Further, in the illustrated embodiment of FIG. 3, the at least one heating element 135 is thermally coupled to the heater block 126.

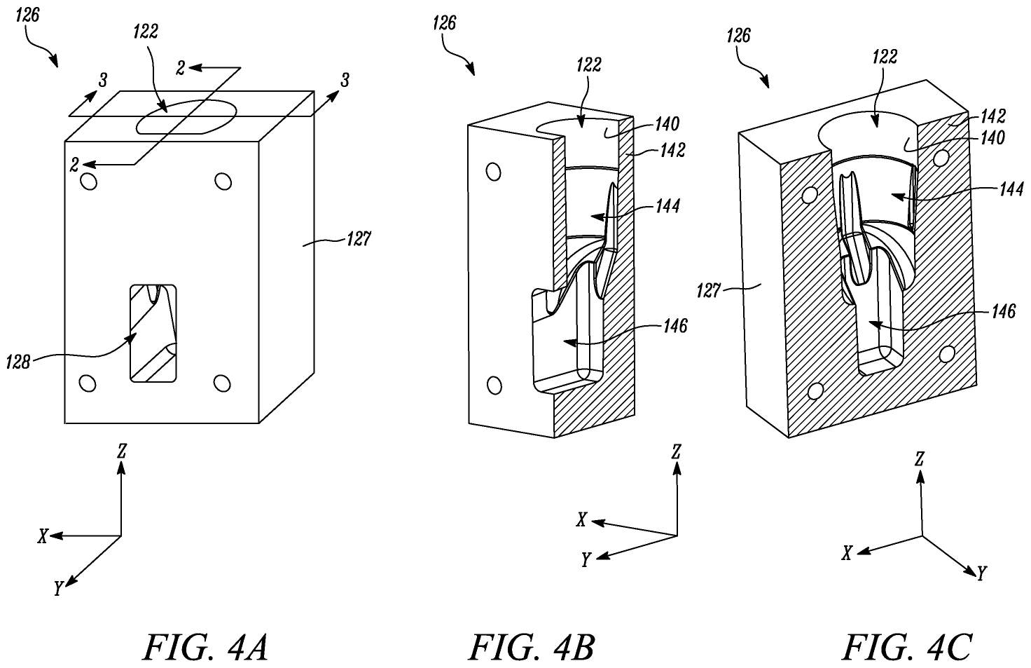

FIGS. 4A-4C illustrate the heater block 126 of the reading apparatus 110 according to an embodiment of the present disclosure. Specifically, FIG. 4A illustrates a schematic front perspective view of the heater block 126, FIG. 4B illustrates a schematic cross-sectional perspective view of the heater block 126 taken along a line 2-2 of FIG. 4A, and FIG. 4C illustrates a schematic cross-sectional perspective view of the heater block 126 taken along a line 3-3 of FIG. 4A.

Referring to FIGS. 3 and 4A-4C, the heater block 126 includes a shape that may be any one of a cube, a cuboid, a sphere, and so forth. For example, in the illustrated embodiment of FIGS. 4A-4C, the heater block 126 includes a cuboid shape and a side surface 127. Further, in the illustrated embodiment of FIGS. 4A-4C, the heater block 126 includes a cavity 128. The cavity 128 may at least partially house one or more elements of the reading apparatus 110.

The heater block 126 may distribute heat from the heating element 135 to the sterilization indicator 10. The heater block 126 is preferably at least partially formed from thermally conductive materials, such as metals (e.g., steel, copper, aluminum), thermally conductive polymers, ceramics, or combinations thereof (including overlaid over non-thermally conductive polymers). In at least one embodiment, the heater block 126 may be non-thermally conductive and formed from polymeric materials. However, it may be noted that the heater block 126 is optional. In other words, in some embodiments, the reading apparatus 110 may not include the heater block 126.

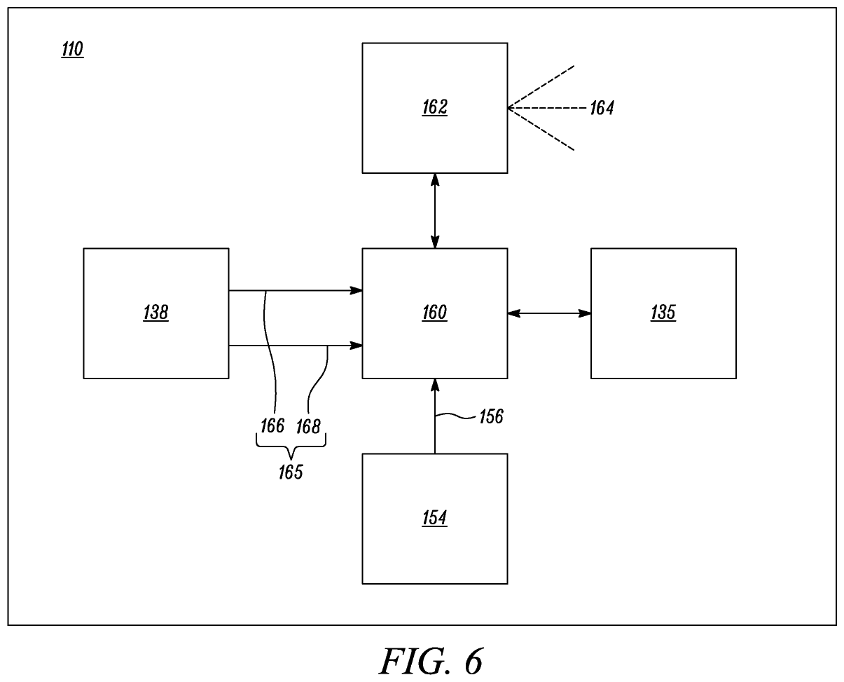

In the illustrated embodiment of FIG. 3, the reading apparatus 110 further includes at least one temperature sensor 154. The at least one temperature sensor 154 may be thermally coupled to the heater block 126. In the illustrated embodiment of FIG. 3, the at least one temperature sensor 154 is separate from the at least one heating element 135. However, in some other embodiments, the at least one heating element 135 may have the temperature sensor 154 embedded therein. In some embodiments, the at least one temperature sensor 154 is configured to generate one or more signals 156 (shown in FIG. 6) indicative of a temperature of the heater block 126. The at least one temperature sensor 154 and the at least one heating element 135 may ensure that the heat applied by the at least one heating element 135 to the coupling portion 134 (e.g., the heater block 126 forming the coupling portion 134) may achieve one or more preset temperatures in a closed-loop heating system.

The reading apparatus 110 further includes at least one sensor 138 configured to sense at least one parameter associated with the sterilization indicator 10 received at least partially within the at least one well 122. The at least one sensor 138 is shown to be disposed adjacent to the second chamber 146 in FIG. 3. In some embodiments, the at least one sensor 138 may be partially disposed within the cavity 128 (shown in FIG. 4A) of the heater block 126. However, the at least one sensor 138 may be disposed at any suitable position proximal to the at least one well 122. The at least one sensor 138 is further configured to generate one or more response signals 165 (shown in FIG. 6) upon sensing the at least one parameter.

The at least one parameter associated with the sterilization indicator 10 may be indicative of, for example, a position of the sterilization indicator 10 with respect to the at least one well 122, an activation of the sterilization indicator 10, and the like. In some embodiments, the at least one sensor 138 includes at least one of a color sensor, a proximity sensor, a pressure sensor, an optical sensor, and an electromechanical switch.

Referring to FIGS. 1, 2, and 3, in some embodiments, the at least one parameter associated with the sterilization indicator 10 includes a change in a viability of the spores 30 of the sterilization indicator 10. In some embodiments, determination of the change in the viability of the spores 30 of the sterilization indicator 10 includes the activation of the sterilization indicator 10 by the mixing of the substance 24 with the spores 30. As discussed above, mixing of the substance 24 with the spores 30 may be achieved via use of the ampoule crusher 26. In some embodiments, the activation of the sterilization indicator 10 may be detected by the color sensor and/or the optical sensor. Specifically, in some embodiments, the at least one sensor 138 is further configured to determine the activation of the sterilization indicator 10 by sensing at least one of an absorbance, a luminescence, and a turbidity of the substance 24 mixed with the spores 30. In some cases, a flexible element, such as foam, may be disposed on the wall 142 of the at least one well 122 to block ambient light from reaching the at least one sensor 138 to improve determination of the activation of the sterilization indicator 10.

In some embodiments, the at least one parameter associated with the sterilization indicator 10 includes an insertion of the sterilization indicator 10 within the at least one well 122. In some embodiments, the insertion of the sterilization indicator 10 within the at least one well 122 may be detected by the proximity sensor and/or the pressure sensor. In some examples, the insertion of the sterilization indicator 10 within the at least one well 122 may be confirmed by use of the electromechanical switch.

In the illustrated embodiment of FIG. 3, the reading apparatus 110 further includes at least one excitation source 136 configured to excite the substance 24 of the sterilization indicator 10. The at least one excitation source 136 may be any light source that causes the substance 24 of the sterilization indicator 10 to emit fluorescence. The at least one excitation source 136 is shown to be disposed adjacent to the second chamber 146 in FIG. 3. In some embodiments, the at least one excitation source 136 may be partially disposed within the cavity 128 (shown in FIG. 4A) of the heater block 126. In some cases, the at least one excitation source 136 may be configured to illuminate the growth chamber 16 of the sterilization indicator 10.

The at least one excitation source 136 may emit ultraviolet (UV) electromagnetic radiation. In at least one embodiment, the UV electromagnetic radiation may have a wavelength ranging from 10 nanometers (nm) to 400 nm. However, wavelengths ranging between 100 nm and 280 nm (i.e., UV-C) may be germicidal and interfere with the growth of the spores 30. Thus, the wavelengths produced by the at least one excitation source 136 may range between 300 nm and 400 nm.

The at least one excitation source 136 may be any device that produces a UV light, such as incandescent bulbs, lasers, light emitting diodes (LEDs), and the like. In some embodiments, the at least one excitation source 136 includes at least one of an ultraviolet light emitting diode (UV LED), an ultraviolet laser (UV laser), and a white light source. In some embodiments, the white light source and the at least one sensor 138 may be additionally or alternatively used for determining the activation of the sterilization indicator 10.

In the illustrated embodiment of FIG. 3, the reading apparatus 110 further includes a printed circuit board 130 and a spacing device 132.

The printed circuit board 130 may include the excitation source 136 and the at least one sensor 138. The printed circuit board 130 may be rigid and planar. However, in some embodiments, the printed circuit board 130 may be flexible. Further, in some embodiments, each well 122 from the plurality of wells 122 may have a separate excitation source 136 and a separate sensor 138.

Specifically, in some embodiments, the at least one sensor 138 includes a plurality of sensors 138. In such embodiments, each sensor 138 is configured to sense the at least one parameter associated with the sterilization indicator 10 received at least partially within a corresponding well 122 from the plurality of wells 122. Further, in some examples, the excitation source 136 includes a plurality of excitation sources 136. Each excitation source 136 from the plurality of excitation sources 136 may be configured to excite the substance 24 of the sterilization indicator 10. In at least one embodiment, the printed circuit board 130 may be continuous such that the excitation source 136, and the at least one sensor 138 corresponding to one well 122 from the plurality of wells 122 are on the same printed circuit board 130 as the excitation source 136 and the sensor 138 of an adjacent well 122 from the plurality of wells 122. In some embodiments, the printed circuit board 130 may be arranged substantially parallel to the well axis 129.

Further, in the illustrated embodiment of FIG. 3, the spacing device 132 may maintain positioning and alignment between electronic elements on the printed circuit board 130, and the sterilization indicator 10 or the heater block 126. The spacing device 132 may be formed from a rigid material, such as polycarbonate or metal. In at least one embodiment, the spacing device 132 is formed from a light absorbing or non-reflective material to minimize interference by ambient light. For example, the spacing device 132 may have a matte finish so as to not reflect light. The spacing device 132 may be black or gray in color. Thus, the spacing device 132 may have a reflectivity no greater than 10 percent, or no greater than 5 percent for light having a wavelength ranging between 400 nm and 700 nm. In at least one embodiment, the spacing device 132 may be mechanically coupled to a portion of the heater block 126 and/or the printed circuit board 130. In the illustrated embodiment of FIG. 3, the spacing device 132 is shown as being disposed adjacent to a base portion of the heater block 126. In at least one embodiment, the spacing device 132 may align the sterilization indicator 10 with the heater block 126 and may form a portion of the at least one well 122. In some embodiments, the at least one well 122 may be formed from both the heater block 126 and the spacing device 132.

In at least one embodiment, the second chamber 146 may be proximal to the spore carrier 28 of the sterilization indicator 10. In at least one embodiment, the at least one heating element 135 may be proximal to the second chamber 146 of the well 122, such that the spores 30 and the substance 24 are heated locally. The second chamber 146 may form an optical path 148 from the excitation source 136 to the growth chamber 16 (containing the spores 30) of the sterilization indicator 10. Fluorescent output from the substance 24 in the growth chamber 16 of the sterilization indicator 10 may be further received by the at least one sensor 138.

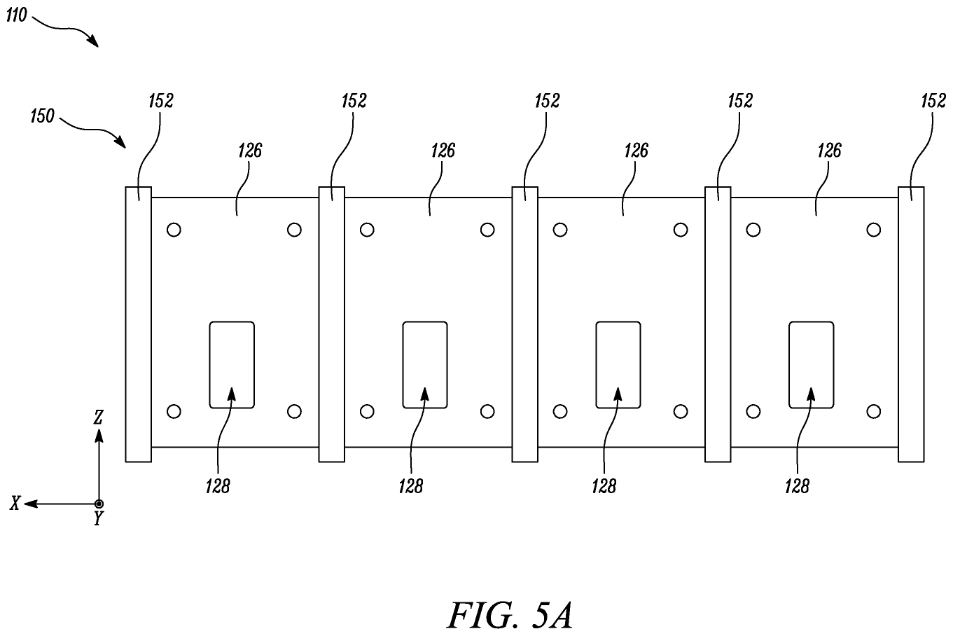

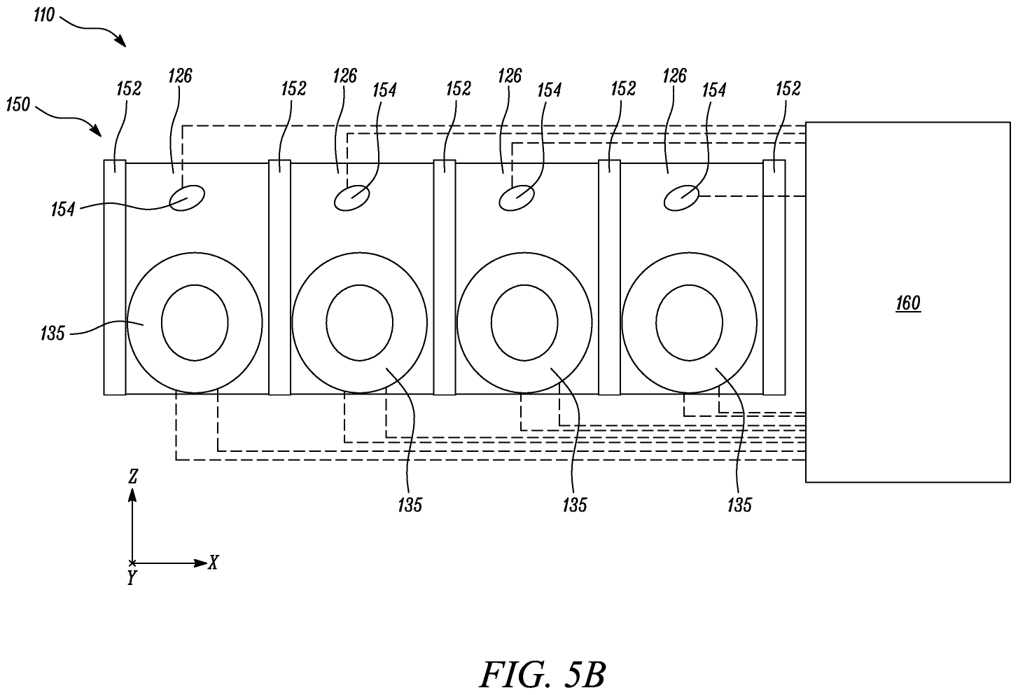

FIGS. 5A and 5B illustrate the reading apparatus 110 according to an embodiment of the present disclosure with some elements of the reading apparatus 110 not shown for illustrative purposes. Specifically, FIG. 5A illustrates a schematic front view of the reading apparatus 110, and FIG. 5B illustrates a schematic rear view of the reading apparatus 110 of FIG. 5A.

In the illustrated embodiment of FIGS. 5A and 5B, the reading apparatus 110 includes a heater block assembly 150. The reading apparatus 110 further includes a processor 160 (shown in FIG. 5B).

Referring to FIGS. 2. 3. 5A, and 5B, in some embodiments, the one heater block 126 includes a plurality of heater blocks 126. Further, each of the plurality of heater blocks 126 forms the coupling portion 134 of a corresponding well 122 from the plurality of wells 122.

Specifically, in the illustrated embodiment of FIGS. 5A and 5B, the plurality of heater blocks 126 includes four of the heater blocks 126 disposed adjacent to each other. In the illustrated embodiment of FIGS. 5A and 5B, the reading apparatus 110 further includes an insulating layer 152 disposed between at least two adjacent heater blocks 126 from the plurality of heater blocks 126 to thermally isolate the at least two adjacent heater blocks 126 from each other. The insulating layer 152 may be disposed between the at least two adjacent heater blocks 126, such that the insulating layer 152 may substantially restrict heat transfer from one heater block 126 from the at least two adjacent heater blocks 126 to another heater block 126 from the at least two adjacent heater blocks 126. As shown in FIGS. 5A and 5B, the heater block assembly 150 may include the plurality of heater blocks 126 alternating with a plurality of insulating layers 152.

In the illustrated embodiment of FIGS. 5A and 5B, one insulating layer 152 is disposed between the at least two adjacent heater blocks 126 from the plurality of heater blocks 126. However, in some other embodiments, more than one insulating layers 152 may be disposed between the at least two adjacent heater blocks 126, depending upon desired application attributes. In some embodiments, the insulating layer 152 may include a material including, but not limited to, a fiberglass, a mineral wool, a cellulose insulation material, a polyurethane foam, or a polystyrene layer (commonly known as Styrofoam).

As discussed above, the reading apparatus 110 further includes the processor 160. The processor 160 is communicably coupled to the at least one heating element 135 and the at least one sensor 138. In the illustrated embodiment if FIG. 5B, the at least one temperature sensor 154 is communicably coupled to the processor 160.

Specifically, in the illustrated embodiment of FIG. 5B, the at least one heating element 135 includes a plurality of heating elements 135. Further, each heating element 135 is thermally coupled to a corresponding well 122 from the plurality of wells 122. In the illustrated embodiment of FIG. 5B, the at least one temperature sensor 154 includes a plurality of temperature sensors 154. Each temperature sensor 154 may be thermally coupled to the heater block 126 of a corresponding well 122 from the plurality of wells 122. Furthermore, in some embodiments, each of the plurality of sensors 138, the plurality of heating elements 135, and the plurality of temperature sensors 154 are communicably coupled to the processor 160.

FIG. 6 illustrates a schematic block diagram of the reading apparatus 110 according to an embodiment of the present disclosure. As shown in FIG. 6, the reading apparatus 110 includes the processor 160 communicably coupled to the at least one heating element 135 and the at least one sensor 138. In the illustrated embodiment of FIG. 6, the reading apparatus 110 further includes an alert unit 162 and the at least one temperature sensor 154. Further, the processor 160 is further communicably coupled to the alert unit 162 and the at least one temperature sensor 154. As discussed above, in some embodiments, the at least one temperature sensor 154 is configured to generate the one or more signals 156 indicative of the temperature of the heater block 126. In some embodiments, the processor 160 is further configured to control the at least one heating element 135 based on the one or more signals 156 received from the at least one temperature sensor 154.

In some embodiments, the printed circuit board 130 (shown in FIG. 3) may include the processor 160. However, in some other embodiments, the processor 160 may be an external processing unit of a device such as a central processing unit (CPU) of a desktop or a laptop and may be communicably coupled to the printed circuit board 130.

Referring to FIGS. 1-3, and 6, as discussed above, the at least one sensor 138 is configured to sense the at least one parameter associated with the sterilization indicator 10 received at least partially within the at least one well 122. Further, as discussed above, the at least one sensor 138 is further configured to generate the one or more response signals 165 upon sensing the at least one parameter. The one or more response signals 165 include a first response signal 166 and a second response signal 168.

In some embodiments, the first response signal 166 is indicative of the activation of the sterilization indicator 10. In some other embodiments, the first response signal 166 is indicative of the insertion of the sterilization indicator 10 into the at least one well 122. As discussed above, in some embodiments, the at least one heating element 135 includes the plurality of heating elements 135 (shown in FIG. 5B) and the at least one sensor 138 includes the plurality of sensors 138. In some embodiments, the processor 160 is further configured to independently control each heating element 135 based at least on the first response signal 166 received from a corresponding sensor 138 from the plurality of sensors 138.

In some embodiments, the second response signal 168 is indicative of a detection of a fluorescence by the substance 24. Moreover, in some embodiments, the processor 160 is further configured to generate an alert 164 upon the detection of the fluorescence by the substance 24. The alert unit 162 may produce the alert 164 generated by the processor 160. The alert 164 may be an audible alert, a visual alert, and/or a haptic alert. In some cases, the processor 160 may be configured to generate a notification upon detection of the activation of the sterilization indicator 10 and/or the insertion of the sterilization indicator 10 within the at least one well 122. The notification may be similar to or different from the alert 164. The notification may be an audible notification, a visual notification, and/or a haptic notification. The alert unit 162 may produce the notification generated by the processor 160.

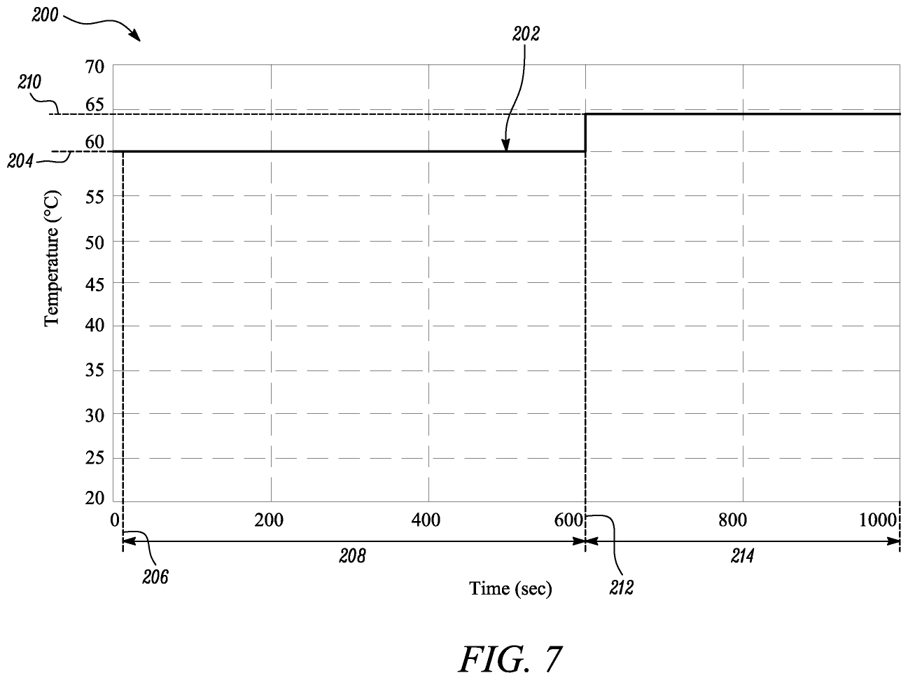

FIG. 7 illustrates a graph 200 representing temperature (in ° C.) on an axis of ordinates (Y-axis) and time (in seconds) on an axis of abscissas (X-axis). The graph 200 includes a curve 202 depicting a variation of a desired temperature of the coupling portion 134 of the at least one well 122 (shown in FIG. 3) with respect to time.

Referring to FIGS. 3, 6, and 7, the processor 160 is configured to control the at least one heating element 135 to achieve a first preset temperature 204 of at least the coupling portion 134 of the at least one well 122 from a first instance of time 206 after receiving the first response signal 166 from the at least one sensor 138. As discussed above, in some embodiments, the first response signal 166 may be indicative of the activation of the sterilization indicator 10 (also shown in FIG. 1) and/or may be indicative of the insertion of the sterilization indicator 10 into the at least one well 122. Therefore, in some embodiments, at the first instance of time 206, the sterilization indicator 10 may be activated and/or the sterilization indicator 10 may be inserted into the at least one well 122.

In some embodiments, the processor 160 is further configured to determine the first instance of time 206 after a predetermined time interval from receiving the first response signal 166. As depicted by the graph 200, the first instance of time 206 is about 15 seconds after receiving the first response signal 166. That is, as depicted by the graph 200, the processor 160 receives the first response signal 166 at 0 seconds, and the predetermined time interval from receiving the first response signal 166 is about 15 seconds. However, in some other cases, the first instance of time 206 may correspond to an instance of time when the first response signal 166 is received. In other words, in some cases, the processor 160 may be configured to control the at least one heating element 135 to achieve the first preset temperature 204 of at least the coupling portion 134 of the at least one well 122 upon receiving the first response signal 166 from the at least one sensor 138.

The processor 160 is further configured to control the at least one heating element 135 to achieve a second preset temperature 210 of at least the coupling portion 134 of the at least one well 122 upon receiving a second response signal 168 from the at least one sensor 138, or after a predetermined time duration 208 from the first instance of time 206. In other words, the processor 160 may be configured to control the at least one heating element 135 to achieve the second preset temperature 210 of at least the coupling portion 134 upon receiving the second response signal 168, or may be configured to automatically control the at least one heating element 135 to achieve the second preset temperature 210 of at least the coupling portion 134 after the predetermined time duration 208 from the first instance of time 206. In some embodiments, the processor 160 is further configured to control the at least one heating element 135 to maintain the second preset temperature 210 of at least the coupling portion 134 of the at least one well 122 for a second time duration 214. In some embodiments, the second time duration 214 is from about 2 hours to about 7 days.

For example, in some cases, the detection of the fluorescence by the substance 24 may be achieved prior to an end of the predetermined time duration 208. Therefore, the processor 160 may receive the second response signal 168 indicative of the detection of the fluorescence by the substance 24 prior to an end of the predetermined time duration 208. The processor 160 may control the at least one heating element 135 to achieve the second preset temperature 210 of at least the coupling portion 134 of the at least one well 122 upon receiving the second response signal 168 from the at least one sensor 138, prior to the end of the predetermined time duration 208.

The second preset temperature 210 is different from the first preset temperature 204. In some embodiments, the second preset temperature 210 is greater than the first preset temperature 204. In some other embodiments, the second preset temperature 210 may be less than the first preset temperature 204. In some embodiments, the first preset temperature 204 is from about 50° C., to about 70° C. In some embodiments, the second preset temperature 210 is from about 55° C., to about 75° C. In some embodiments, the first preset temperature 204 is about 60° C., and the second preset temperature 210 is about 64° C. However, it may be noted that the second preset temperature 210 and the first preset temperature 204 can be selected based on desired application attributes (e.g., a type of the sterilization indicator 10 (shown in FIG. 1)).

Such control of the at least one heating element 135 may optimize different phases of incubation of the sterilization indicator 10 (shown in FIG. 1). In one example, the first preset temperature 204 may speed up and facilitate a first phase of incubation of the spores 30 of the sterilization indicator 10 (e.g., an α-glucosidase reaction phase). Furthermore, the second preset temperature 210 may speed up and facilitate a second phase of incubation of the spores 30 of the sterilization indicator 10 (e.g., a growth phase). Therefore, the reading apparatus 110 may effectively control the temperature of the coupling portion 134 to reduce the time taken to read the sterilization indicator 10 and obtain a result indicative of an effectiveness of a sterilization process.

The processor 160 may control the at least one heating element 135 to achieve the second preset temperature 210 of at least the coupling portion 134 of the at least one well 122 at a second instance of time 212. In some cases, the second instance of time 212 may be after the predetermined time duration 208 from the first instance of time 206. In the graph 200, the predetermined time duration 208 is about 10 minutes. However, in some embodiments, the predetermined time duration 208 is from about 5 minutes to about 15 minutes. In some other cases, the second instance of time 212 may occur upon receiving the second response signal 168 from the at least one sensor 138. In other words, at the second instance of time 212, the processor 160 may receive the second response signal 168 from the at least one sensor 138.

In some embodiments, the processor 160 is further configured to control the at least one heating element 135 to maintain a temperature of the heater block 126 at the first preset temperature 204 for the predetermined time duration 208. In some embodiments, the processor 160 is further configured to control the at least one heating element 135 to change the temperature of the heater block 126 to the second preset temperature 210 from the first preset temperature 204 after the predetermined time duration 208.

In some embodiments, the processor 160 is further configured to set a temperature setpoint of the heater block 126 to the first preset temperature 204 for the predetermined time duration 208. In some embodiments, the processor 160 is further configured to set the temperature setpoint of the heater block 126 to the second preset temperature 210 after the predetermined time duration 208. In some other embodiments, the processor 160 is further configured to set the temperature setpoint of the heater block 126 to the second preset temperature 210 upon receiving the second response signal 168. In some embodiments, the processor 160 is further configured to control the at least one heating element 135 in order to achieve the temperature setpoint of the heater block 126. In other words, the temperature setpoint (i.e., the first and second preset temperatures 204. 210) may be achieved by controlling the at least one heating element 135.

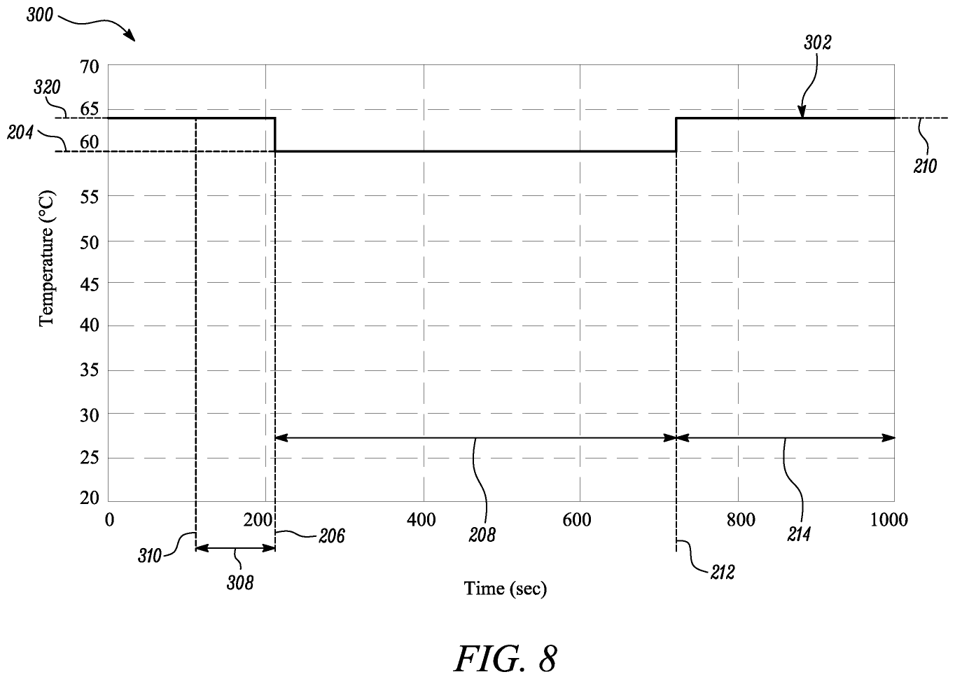

FIG. 8 illustrates a graph 300 representing temperature (in ° C.) on an axis of ordinates (Y-axis) and time (in seconds) on an axis of abscissas (X-axis). The graph 300 includes a curve 302 depicting a variation of a desired temperature of the coupling portion 134 of the at least one well 122 (shown in FIG. 3) with respect to time.

Referring to FIGS. 3, 6, and 8, in some embodiments, the processor 160 is further configured to control the at least one heating element 135 to achieve a third preset temperature 320 of at least the coupling portion 134 of the at least one well 122 prior to receiving the first response signal 166.

The third preset temperature 320 is different from the first preset temperature 304. Specifically, in some embodiments, the third preset temperature 320 is greater than the first preset temperature 204. Further, in some embodiments, each of the second preset temperature 210 and the third preset temperature 320 is different from the first preset temperature 204.

As depicted by the curve 302, in some embodiments, the third preset temperature 320 is substantially equal to the second preset temperature 210. Specifically, in the graph 300, the first preset temperature 204 is about 60° C. and each of the second preset temperature 210 and the third preset temperature 320 is about 64° C. However, as discussed above, in some embodiments, the first preset temperature 204 is from about 50°° C. to about 70° C. and the second preset temperature 210 is from about 55° C. to about 75° C. In some embodiments, the third preset temperature 320 is from about 50° C. to about 70° C. In some embodiments, the third preset temperature 320 is about 64° C.

In some examples, the third preset temperature 320 may be greater than the first preset temperature 204. Therefore, at least the coupling portion 134 may be preheated to the third preset temperature 320. Preheating the coupling portion 134 to the third preset temperature 320 may increase heat flow to the sterilization indicator 10 to speed up and facilitate a phase of incubation of the spores 30 (shown in FIG. 1) of the sterilization indicator 10. Consequently, the reading apparatus 110 may further reduce the time taken to read the sterilization indicator 10 and obtain the result indicative of the effectiveness of the sterilization process.

In some embodiments, the processor 160 is further configured to set the temperature setpoint of the heater block 126 of the at least one well 122 to the third preset temperature 320 prior to receiving the first response signal 166. In some embodiments, the processor 160 is further configured to set the temperature setpoint of the heater block 126 of the at least one well 122 to the first preset temperature for the predetermined time duration 208 after a predetermined time interval 308 from receiving the first response signal 166. In the graph 300, the predetermined time interval 308 begins from a third instance of time 310 and ends at the first instance of time 206. Thus, the processor 160 may receive the first response signal 166 at the third instance of time 310. In some embodiments, the processor 160 is further configured to determine the first instance of time 206 after the predetermined time interval 308 from receiving the first response signal 166. In some embodiments, the predetermined time interval 308 is from about 15 seconds to about 120 seconds

Moreover, in some embodiments, the processor 160 is further configured to set the temperature setpoint of the heater block 126 of the at least one well 122 to the second preset temperature 210 after the predetermined time duration 208. The processor 160 is further configured control the at least one heating element 135 in order to achieve the temperature setpoint of the heater block 126.

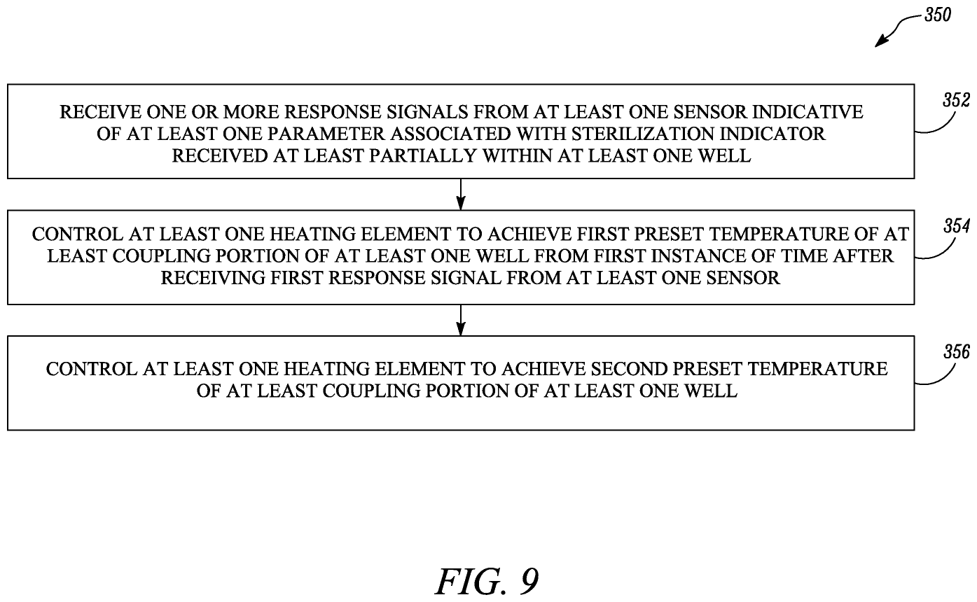

FIG. 9 illustrates a flowchart depicting various steps of a method 350 of controlling a sterilization indicator reading apparatus according to an embodiment of the present disclosure. The sterilization indicator reading apparatus has a housing, at least one well formed from a portion of the housing, at least one heating element thermally coupled to a coupling portion of the at least one well, at least one sensor. and a processor communicably coupled to the at least one heating element and the at least one sensor. In some embodiments, the method 350 may be implemented by the processor 160 (shown in FIG. 6) of the reading apparatus 110. The method 350 will be further described with reference to FIGS. 1-8.

At step 352, the method 350 includes receiving, via the processor, one or more response signals from the at least one sensor indicative of at least one parameter associated with a sterilization indicator received at least partially within the at least one well. The sterilization indicator has spores and a substance fluorescently responsive to a spore viability. The spores are responsive to an environmental condition in a sterilizer. For example, the method 350 may include receiving, via the processor 160, the one or more response signals 165 from the at least one sensor 138 indicative of the at least one parameter associated with the sterilization indicator 10 received at least partially within the at least one well 122.

In some embodiments, the at least one parameter associated with the sterilization indicator includes at least one of an activation of the sterilization indicator by a mixing of the substance with the spores and an insertion of the sterilization indicator within the at least one well. In some embodiments, the method 350 further includes determining, via the at least one sensor, the activation of the sterilization indicator by sensing at least one of an absorbance, a luminescence, and a turbidity of the substance mixed with the spores. For example, the method 350 may include determining, via the at least one sensor 138, the activation of the sterilization indicator 10 by sensing at least one of the absorbance, the luminescence, and the turbidity of the substance 24 mixed with the spores 30.

In some embodiments, the method 350 further includes determining, via the processor, the first instance of time after a predetermined time interval from receiving the first response signal. For example, the method 350 may further include determining, via the processor 160, the first instance of time 206 after the predetermined time interval 308 from receiving the first response signal 166. In some embodiments, the predetermined time interval is from about 15 seconds to about 120 seconds. For example, the predetermined time interval 308 may be from about 15 seconds to about 120 seconds.

At step 354, the method 350 further includes controlling, via the processor, the at least one heating element to achieve a first preset temperature of at least the coupling portion of the at least one well from a first instance of time after receiving a first response signal from the at least one sensor. For example, the method 350 may include controlling, via the processor 160, the at least one heating element 135 to achieve the first preset temperature 204 of at least the coupling portion 134 of the at least one well 122 from the first instance of time 206 after receiving the first response signal 166 from the at least one sensor 138.

In some embodiments, the first response signal is indicative of at least one of the activation of the sterilization indicator and the insertion of the sterilization indicator into the at least one well. For example, the first response signal 166 may be indicative of at least one of the activation of the sterilization indicator 10 and the insertion of the sterilization indicator 10 into the at least one well 122.

In some embodiments, the method 350 further includes controlling, via the processor, the at least one heating element to maintain a temperature of the at least one heater block at the first preset temperature for the predetermined time duration. For example, the method 350 may further include controlling, via the processor 160, the at least one heating element 135 to maintain the temperature of the heater block 126 at the first preset temperature 204 for the predetermined time duration 208.

In some embodiments, the method 350 further includes setting, via the processor, a temperature setpoint of the at least one heater block to the first preset temperature for the predetermined time duration. For example, the method 350 may further include setting, via the processor 160, the temperature setpoint of the heater block 126 to the first preset temperature 204 for the predetermined time duration 208.

In some embodiments, the method 350 further includes controlling, via the processor, the at least one heating element based on one or more signals received from at least one temperature sensor thermally coupled to at least one heater block forming the coupling portion of the at least one well. For example, the method 350 may include controlling, via the processor 160, the at least one heating element 135 based on the one or more signals 156 received from the at least one temperature sensor 154 thermally coupled to the heater block 126 forming the coupling portion 134 of the at least one well 122.

At step 356, the method 350 further includes controlling, via the processor, the at least one heating element to achieve a second preset temperature of at least the coupling portion of the at least one well upon receiving a second response signal from the at least one sensor, or after a predetermined time duration from the first instance of time. For example, the method 350 may include controlling, via the processor 160, the at least one heating element 135 to achieve the second preset temperature 210 of at least the coupling portion 134 of the at least one well 122 upon receiving the second response signal 168 from the at least one sensor 138, or after the predetermined time duration 208 from the first instance of time 206.

The second preset temperature is different from the first preset temperature. The second preset temperature may be greater than or less than the first preset temperature. In some embodiments, the first preset temperature is about 60° C. and the second preset temperature is about 64° C. For example, the first preset temperature 204 may be about 60° C. and the second preset temperature 210 may be about 64° C.

In some embodiments, the second response signal is indicative of a detection of a fluorescence by the substance. For example, the second response signal 168 may be indicative of the detection of the fluorescence by the substance 24. In some embodiments, the method 350 further includes generating, via the processor, an alert upon the detection of the fluorescence by the substance. For example, the method 350 may further include generating, via the processor 160, the alert 164 upon the detection of the fluorescence by the substance 24.

In some embodiments, the predetermined time duration from the first instance of time is from about 5 minutes to about 15 minutes. For example, the predetermined time duration 208 may be from about 5 minutes to about 15 minutes.

In some embodiments, the method 350 further includes, controlling, via the processor, the at least one heating element to maintain the second preset temperature of at least the coupling portion of the at least one well for a second time duration. For example, the method 350 may further include controlling, via the processor 160, the at least one heating element 135 to maintain the second preset temperature 210 of at least the coupling portion 134 of the at least one well 122 for the second time duration 214. In some embodiments, the second time duration is from about 2 hours to about 7 days. For example, the second time duration 214 may be from about 2 hours to about 7 days.

In some embodiments, the method 350 further includes controlling, via the processor, the at least one heating element to change the temperature of the at least one heater block to the second preset temperature from the first preset temperature after the predetermined time duration. For example, the method 350 may further include controlling, via the processor 160, the at least one heating element 135 to change the temperature of the heater block 126 to the second preset temperature 210 from the first preset temperature 204 after the predetermined time duration 208.

In some embodiments, the method 350 further includes setting, via the processor, the temperature setpoint of the at least one heater block to the second preset temperature after the predetermined time duration. For example, the method 350 may further include setting, via the processor 160, the temperature setpoint of the heater block 126 to the second preset temperature 210 after the predetermined time duration 208. In some other embodiments, the method 350 further includes setting, via the processor, the temperature setpoint of the at least one heater block to the second preset temperature upon receiving the second response signal. For example, the method 350 may further include setting, via the processor 160, the temperature setpoint of the heater block 126 to the second preset temperature 210 upon receiving the second response signal 168. In some embodiments, the method 350 further includes controlling, via the processor, the at least one heating element in order to achieve the temperature setpoint of the at least one heater block. For example, in some embodiments, the method 350 further includes controlling, via the processor 160, the at least one heating element 135 in order to achieve the temperature setpoint of the heater block 126.

The temperature setpoint may correspond to a target temperature of the at least one heater block that is to be achieved by controlling the at least one heating element. In some cases, the temperature setpoint is a parameter or a variable stored in a memory accessible by the processor. The value of the parameter is set based on a desired value of the temperature setpoint. For example, when the temperature setpoint is set to the first preset temperature, the value of the parameter is set to the first preset temperature. Similarly, when the temperature setpoint is changed to the second preset temperature from the first preset temperature, the value of the parameter is set to the second present temperature. Dynamically changing the temperature setpoint may allow dynamic control of the temperature of the at least one heater block.

In some embodiments, the method 350 further includes controlling, via the processor, the at least one heating element to achieve a third preset temperature of at least the coupling portion of the at least one well prior to receiving the first response signal, the third preset temperature being different from the first preset temperature. For example, in some embodiments, the method 350 may further include controlling. via the processor 160, the at least one heating element 135 to achieve the third preset temperature 320 of at least the coupling portion 134 of the at least one well 122 prior to receiving the first response signal 166. The third preset temperature 320 may be different from the first preset temperature 204. In some embodiments, the third preset temperature is about 64° C. For example, the third preset temperature 320 may be about 64° C.

### Experimental Results

Experiments were conducted on a conventional reading apparatus and the reading apparatus 110 of the present disclosure for detection of enzymatic growth and for determining a time-to-result (TTR) of each of the conventional reading apparatus and the reading apparatus 110. Referring to FIGS. 1-8, the sterilization indicator 10 was inserted into a well the conventional reading apparatus, and the well 122 of the reading apparatus 110. The sterilization indicator 10 was incubated by the conventional reading apparatus (which used static temperature control) and the reading apparatus 110 (which used dynamic temperature control). A thermocouple was inserted into the sterilization indicator 10 to determine a temperature of the substance 24 of the sterilization indicator 10. The experiments were conducted at an ambient temperature of about 23° C.

The results were determined and were plotted on graphs that are described hereinafter with further reference to FIGS. 1-8.

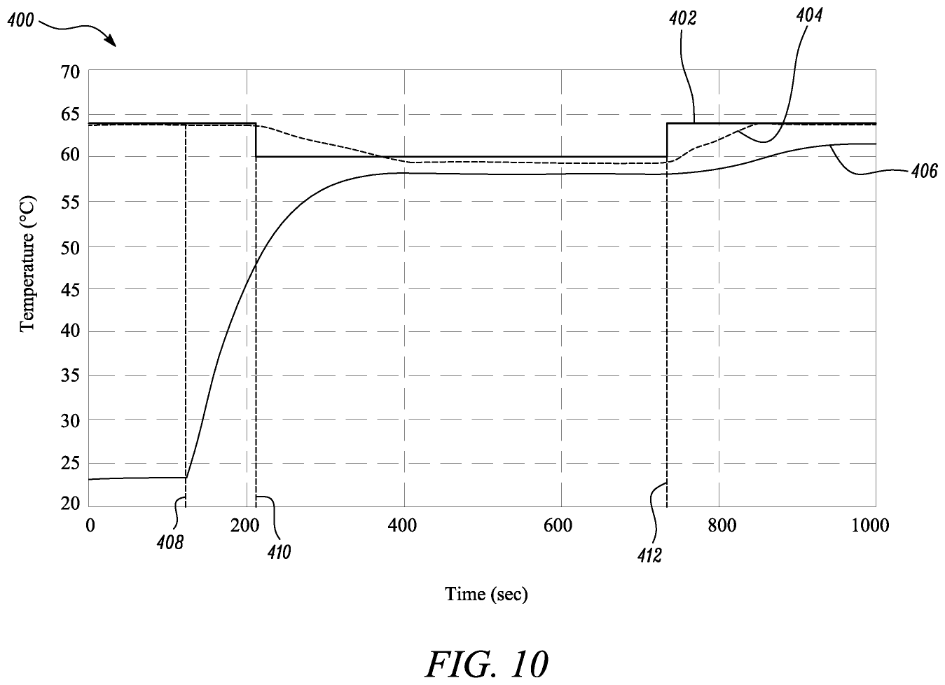

FIG. 10 illustrates a graph 400 representing temperature (in ° C.) on an axis of ordinates (Y-axis) and time (in seconds) on an axis of abscissas (X-axis). The graph 400 includes a first curve 402, a second curve 404, and a third curve 406. The first curve 402 depicts the temperature setpoint of the heater block 126 forming the coupling portion 134 of the at least one well 122 in which the sterilization indicator 10 was received. The second curve 404 depicts a variation of the temperature of the heater block 126. The third curve 406 depicts a variation of the temperature of the substance 24 of the sterilization indicator 10.

As depicted by the first curve 402, initially, the temperature setpoint of the heater block 126 was set to 64° C. At a first time 408, the sterilization indicator 10 was inserted into the well 122 the reading apparatus 110.

At a second time 410, the temperature setpoint of the heater block 126 was set to 60° C. from 64° C. The second time 410 was an instance of time after about 120 seconds from the insertion of the sterilization indicator 10 within the well 122. The temperature setpoint of the heater block 126 was maintained at 60°° C. for about 510 seconds from the second time 410.

At a third time 412 (i.e., after about 510 seconds from the second time 410), the temperature setpoint of the heater block 126 was set to 64° C. from 60° C. The third time 412 was an instance of time in which a fluorescence emitted by the substance 24 of the sterilization indicator 10 was detected.

Further, as depicted by the second curve 404, the temperature of the heater block 126 was about 64° C. up until the second time 410 (as the temperature setpoint was set to 64° C. up until the second time 410). The temperature of the heater block 126 gradually decreased and achieved a steady state at about 59° C. after about 190 seconds from the second time 410. The temperature of the heater block 126 remained at the steady state at about 59° C. for about 320 seconds after the temperature of the heater block 126 gradually decreased to about 59° C. from 64° C. (i.e., for about 320 seconds from the second time 410). After the third time 412, the temperature of the heater block 126 gradually increased to about 64° C. in about 120 seconds from the third time 412.

As depicted by the third curve 406, before the first time 408 (i.e., before the sterilization indicator 10 was inserted into the well 122), the sterilization indicator 10 was at the ambient temperature (i.e., about 23° C.). After the first time 408 (i.e., after the sterilization indicator 10 was inserted into the well 122), the temperature of the substance 24 gradually increased for about 280 seconds from the first time 408 and achieved a steady state at about 58° C. after 280 seconds from the first time 408. The substance 24 then remained at about 58° C. up to the third time 412. After the third time 412, the temperature of the substance 24 gradually increased to about 62° C. (as the temperature setpoint of the heater block 126 was set to 64° C.).

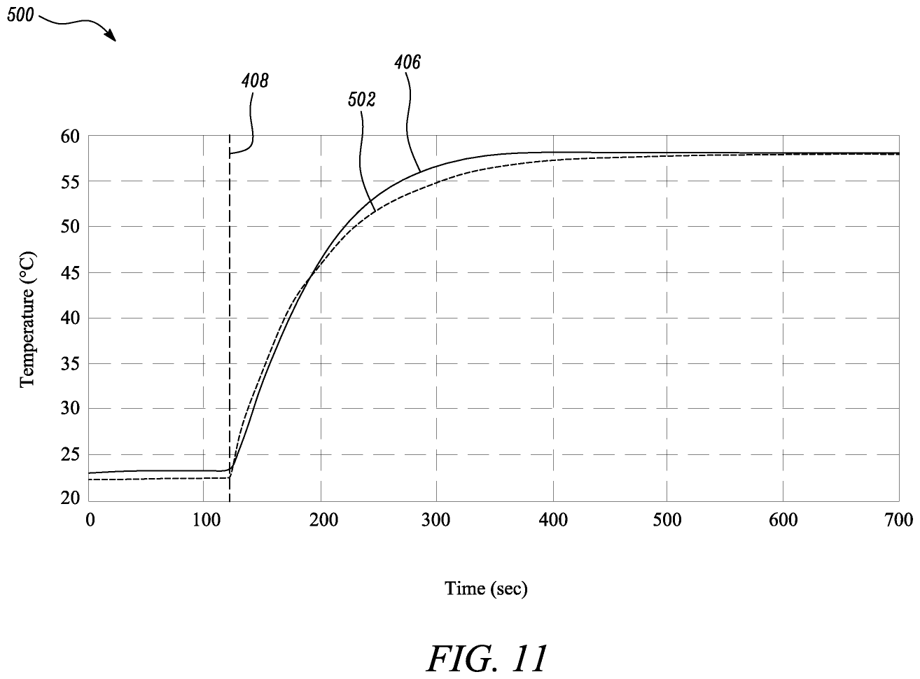

FIG. 11 illustrates a graph 500 representing temperature (in ° C.) on an axis of ordinates (Y-axis) and time (in seconds) on an axis of abscissas (X-axis). The graph 500 includes a curve 502 and a portion of the third curve 406 of FIG. 10. The curve 502 depicts a variation of the temperature of the substance 24 of the sterilization indicator 10 inserted into the well of the conventional reading apparatus (which used the static temperature control). The static temperature control refers to a control of a heating means with a single temperature setpoint throughout an incubation period of the sterilization indicator 10.

After insertion of the sterilization indicator 10 in the conventional reading apparatus (i.e., after the first time 408), the temperature of the substance 24 gradually increased for about 380 seconds and achieved a steady state at about 58° C. after 380 seconds from the first time 408. In contrast, the substance 24 of the sterilization indicator 10 received in the well 122 of the reading apparatus 110 achieved a steady state at about 58° C. after 280 seconds from the first time 408. In conclusion, the substance 24 of the sterilization indicator 10 incubated in the reading apparatus 110 achieved the same temperature (of about 58° C.) about 100 seconds faster than substance 24 of the sterilization indicator 10 incubated in the conventional reading apparatus.

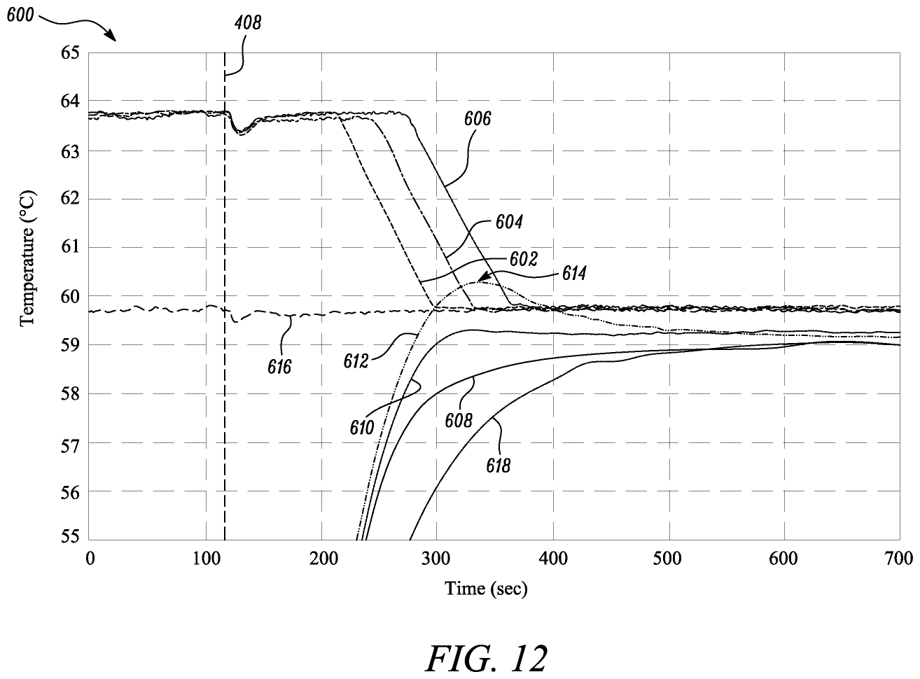

FIG. 12 illustrates a graph 600 representing temperature (in ° C.) on an axis of ordinates (Y-axis) and time (in seconds) on an axis of abscissas (X-axis). The graph 600 includes a first curve 602, a second curve 604, and a third curve 606. Each of the first curve 602, the second curve 604, and the third curve 606 depicts a corresponding variation of the temperature of the heater block 126 of the reading apparatus 110. The variation of the temperature of the heater block 126 was achieved by setting the temperature setpoint of the heater block 126.

The graph 600 further includes a fourth curve 608, a fifth curve 610, and a sixth curve 612. Each of the fourth curve 608, the fifth curve 610, and the sixth curve 612 depicts a variation of the temperature of the substance 24 of the sterilization indicator 10 inserted into the well 122 of the reading apparatus 110. The fourth curve 608 corresponds to the first curve 602, the fifth curve 610 corresponds to the second curve 604, and the sixth curve 612 corresponds to the third curve 606.

As depicted by the first curve 602, the temperature of the heater block 126 was reduced from about 64° C. to about 60° C. after 90 seconds from the first time 408. Further, as depicted by the fourth curve 608, the temperature of the substance 24 gradually increased for about 330 seconds and reached a steady state at about 59°° C. after about 330 seconds from the first time 408.

As depicted by the second curve 604, the temperature of the heater block 126 was reduced from about 64° C. to about 60° C. after 120 seconds from the first time 408. Further, as depicted by the fifth curve 610, the temperature of the substance 24 gradually increased for about 200 seconds and achieved a steady state at about 59°° C. after about 200 seconds from the first time 408.

As depicted by the third curve 606, the temperature of the heater block 126 was reduced from about 64° C. to about 60° C. after 150 seconds from the first time 408. Further, as depicted by the sixth curve 612, an overshoot 614 in the temperature of the substance 24 occurred. The overshoot 614 can be defined as an occurrence in which the temperature of the substance 24 rose above 60° C. The overshoot 614 may cause an inaccurate reading of the sterilization indicator 10 by the reading apparatus 110. However, as depicted by the fourth and fifth curves 608. 610, the overshoot 614 in the temperature of the substance 24 of the sterilization indicator 10 was prevented when the temperature of the heater block 126 was reduced from about 64° C. to about 60° C. in under 120 seconds from the first time 408.

The graph 600 further includes a seventh curve 616 depicting a variation of a temperature a heater block (fixed at a temperature setpoint of about 60° C.) of the conventional reading apparatus, and an eighth curve 618 depicting a variation of the temperature of the substance 24 of the sterilization indicator 10 inserted in the well of the conventional reading apparatus. As depicted by the eighth curve 618, the temperature of the substance 24 gradually increased for about 380 seconds and achieved a steady state at about 59° C. after 380 seconds from the first time 408.

Therefore, a predetermined time interval of about 90 seconds to about 120 seconds between the insertion of the sterilization indicator 10 into the well 122 of the reading apparatus 110 (i.e., the first time 408) and the adjustment of the temperature of the heater block 126 from about 64° C. to about 60° C. resulted in fastest heating of the substance 24 of the sterilization indicator 10 without causing the overshoot 614 in the temperature of the sterilization indicator 10. Further, the conventional reading apparatus took the longest to heat the substance 24 of the sterilization indicator 10 to about 59° C.

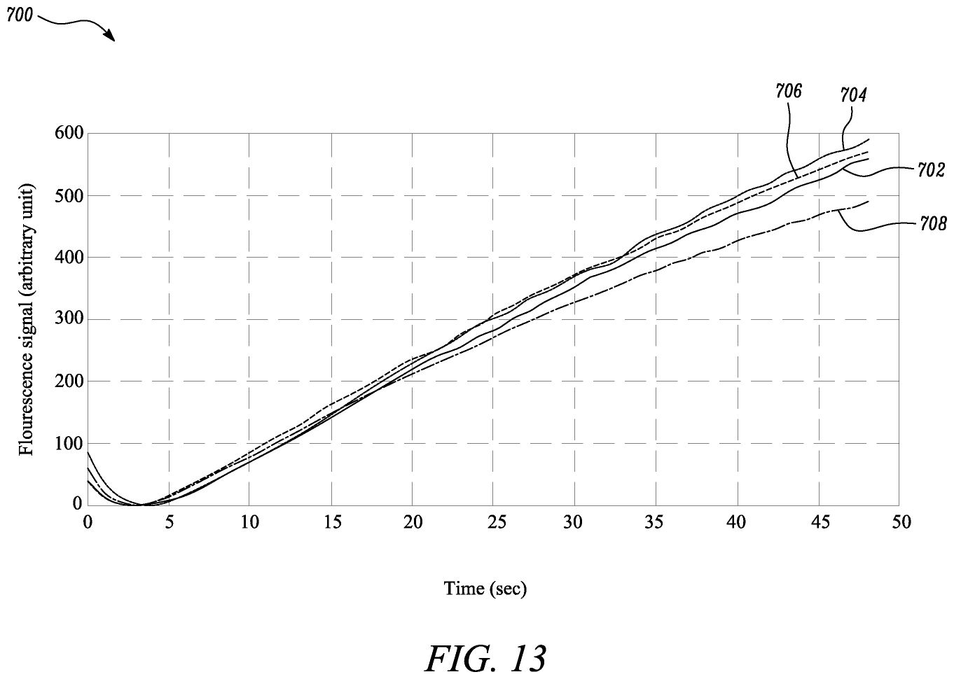

FIG. 13 illustrates a graph 700 representing fluorescence signal (in arbitrary units) on an axis of ordinates (Y-axis) and time (in seconds) on an axis of abscissas (X-axis).

The graph 700 includes a first curve 702, a second curve 704, a third curve 706, and a fourth curve 708. Each of the first curve 702, the second curve 704, the third curve 706, and the fourth curve 708 depicts a corresponding variation of the fluorescence signal generated from fluorescence emitted by the substance 24 of the sterilization indicator 10 incubated in the reading apparatus 110 at a corresponding different temperature. Each of the sterilization indicators 10 incubated in the reading apparatus 110 at the corresponding different temperature had a positive result in the reading apparatus 110.

Specifically, the first curve 702 depicts a variation of the fluorescence signal generated from fluorescence emitted by the substance 24 of the sterilization indicator 10 incubated in the reading apparatus 110 at a temperature of 52° C. The second curve 704 depicts a variation of the fluorescence signal generated from fluorescence emitted by the substance 24 of the sterilization indicator 10 incubated in the reading apparatus 110 at a temperature of 56° C. The third curve 706 depicts a variation of the fluorescence signal generated from fluorescence emitted by the substance 24 of the sterilization indicator 10 incubated in the reading apparatus 110 at a temperature of 60° C. The fourth curve 708 depicts a variation of the fluorescence signal generated from fluorescence emitted by the substance 24 of the sterilization indicator 10 incubated in the reading apparatus 110 at a temperature of 64° C.

As depicted by the graph 700, the sterilization indicators 10 incubated at 56° C. (depicted by the second curve 704) and 60° C. (depicted by the third curve 706) rapidly produced fluorescence that generated fluorescence signals of higher intensities than the sterilization indicators 10 incubated at 52° C. (depicted by the first curve 702) and 64° C. (depicted by the fourth curve 708). The fluorescence signal generated due to fluorescence produced by the substance 24 of the sterilization indicator 10 incubated at 56° C. (depicted by the second curve 704) and the fluorescence signal generated due to fluorescence produced by the substance 24 of the sterilization indicator 10 incubated at 60° C. (depicted by the third curve 706) had the highest amount of signal growth.

In order to determine an optimal temperature for α-glucosidase reaction, four replicates were run at each temperature (54° C., 56° C., 58° C., and 60° C.) to account for well-to-well variability. The samples at each temperature were subtracted and averaged.

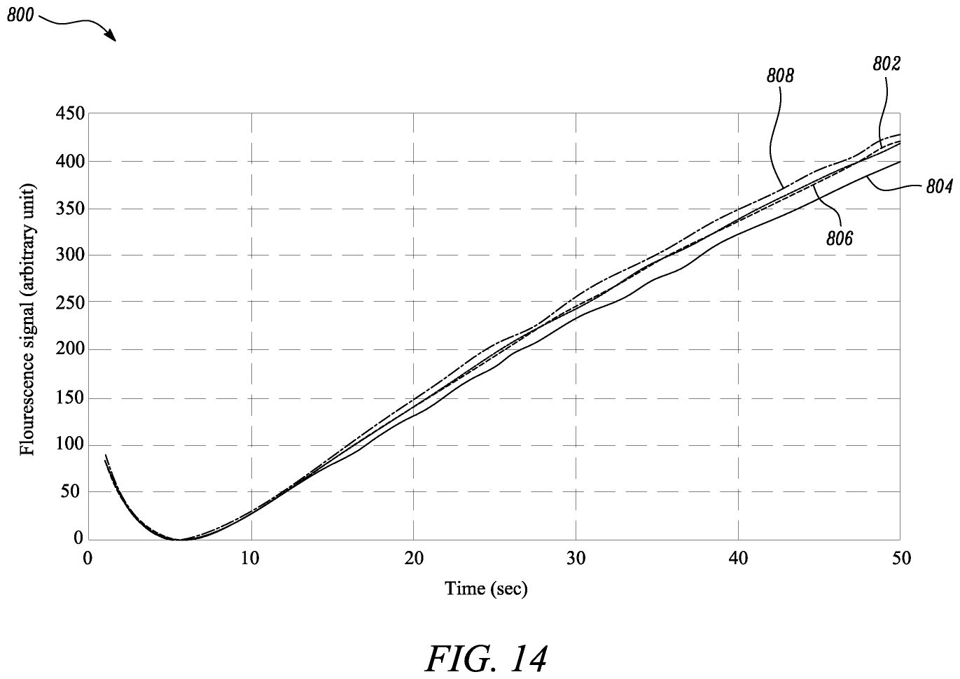

FIG. 14 illustrates a graph 800 representing a fluorescence signal (in arbitrary units) on an axis of ordinates (Y-axis) and time (in seconds) on an axis of abscissas (X-axis).

The graph 800 includes a first curve 802, a second curve 804, a third curve 806, and a fourth curve 808. The first curve 802 depicts a variation of an average fluorescence signal generated from fluorescence emitted by four of the sterilization indicators 10 incubated in the reading apparatus 110 at 54° C. The second curve 804 depicts a variation of an average fluorescence signal generated from the fluorescence emitted by four of the sterilization indicators 10 incubated in the reading apparatus 110 at 56° C. The third curve 806 depicts a variation of an average fluorescence signal generated from the fluorescence emitted by four of the sterilization indicators 10 incubated in the reading apparatus 110 at 58° C. The fourth curve 808 depicts a variation of an average fluorescence signal generated from the fluorescence emitted by four of the sterilization indicators 10 incubated in the reading apparatus 110 at 60° C. Each of the sterilization indicators 10 incubated in the reading apparatus 110 at the corresponding different temperature had the positive result in the reading apparatus 110. As depicted by the graph 800, the sterilization indicators 10 incubated at 60° C. rapidly produced fluorescence that generated average fluorescence signals of higher intensities than the sterilization indicators 10 incubated at 54° C., 56° C., and 58° C.

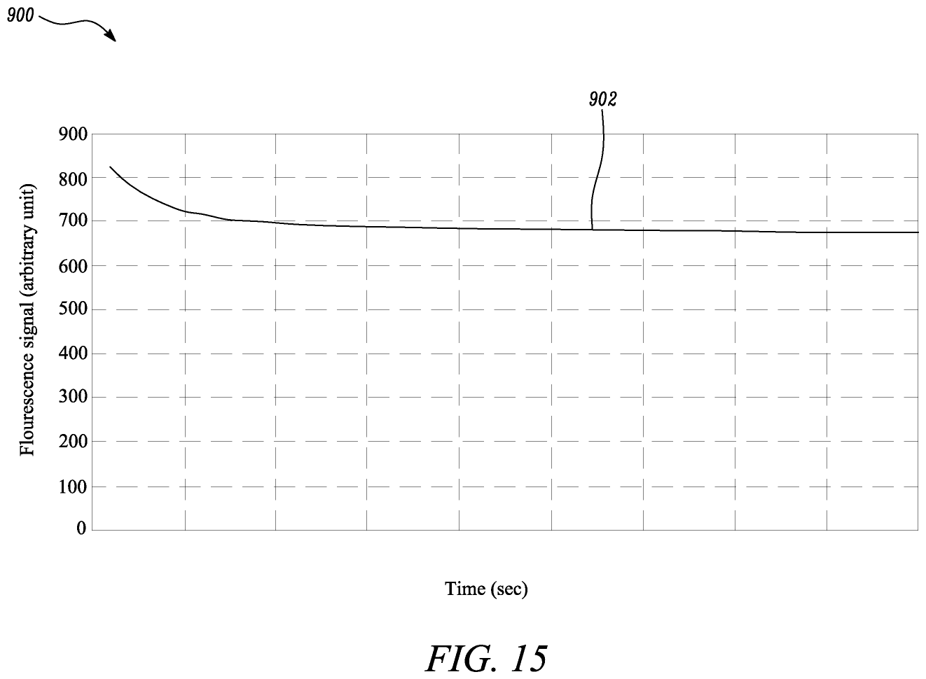

FIG. 15 illustrates a graph 900 representing a fluorescence signal (in arbitrary units) on an axis of ordinates (Y-axis) and time (in seconds) on an axis of abscissas (X-axis). The graph 900 includes a curve 902 depicting a variation of the fluorescence signal generated from fluorescence emitted by the substance 24 of the sterilization indicator 10.

In this case, the sterilization indicator 10 incubated in the reading apparatus 110 at 64° C. had a negative result in the reading apparatus 110. It was determined that at all temperatures (e.g., 54° C., 56° C., 58° C., and 60° C.), the sterilization indicator 10 showed no signal growth over 24-minute incubation when the sterilization process was effective in killing the spores 30.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A sterilization indicator reading apparatus, comprising:
a housing comprising a top portion, and a bottom portion opposite the top portion;
at least one well formed from a portion of the housing, accessible from the top portion, and oriented along a well axis from the top portion to the bottom portion, the at least one well dimensioned to receive at least a portion of a sterilization indicator having spores and a substance fluorescently responsive to a spore viability, wherein the spores are responsive to an environmental condition in a sterilizer;
at least one heating element thermally coupled to a coupling portion of the at least one well;
at least one sensor configured to sense at least one parameter associated with the sterilization indicator received at least partially within the at least one well and generate one or more response signals upon sensing the at least one parameter; and
a processor communicably coupled to the at least one heating element and the at least one sensor, wherein the processor is configured to:
control the at least one heating element to achieve a first preset temperature of at least the coupling portion of the at least one well from a first instance of time after receiving a first response signal from the at least one sensor; and
control the at least one heating element to achieve a second preset temperature of at least the coupling portion of the at least one well:
upon receiving a second response signal from the at least one sensor; or
after a predetermined time duration from the first instance of time;
wherein the second preset temperature is different from the first preset temperature.

2. The sterilization indicator reading apparatus of claim 1, wherein the at least one parameter associated with the sterilization indicator comprises a change in a viability of the spores of the sterilization indicator.

3. The sterilization indicator reading apparatus of claim 2, wherein determination of the change in the viability of the spores of the sterilization indicator comprises an activation of the sterilization indicator by a mixing of the substance with the spores.

4. The sterilization indicator reading apparatus of claim 3, wherein the at least one sensor is further configured to determine the activation of the sterilization indicator by sensing at least one of an absorbance, a luminescence, and a turbidity of the substance mixed with the spores.

5. The sterilization indicator reading apparatus of claim 3, wherein the first response signal is indicative of the activation of the sterilization indicator.

6. The sterilization indicator reading apparatus of claim 1, wherein the at least one parameter associated with the sterilization indicator comprises an insertion of the sterilization indicator within the at least one well.

7. The sterilization indicator reading apparatus of claim 6, wherein the first response signal is indicative of the insertion of the sterilization indicator into the at least one well.

8. The sterilization indicator reading apparatus of claim 1, wherein the at least one sensor comprises at least one of a color sensor, a proximity sensor, a pressure sensor, an optical sensor, and an electromechanical switch.

9. The sterilization indicator reading apparatus of claim 1, further comprising at least one excitation source configured to excite the substance of the sterilization indicator.

10. The sterilization indicator reading apparatus of claim 9, wherein the at least one excitation source comprises at least one of an ultraviolet light emitting diode, an ultraviolet laser, and a white light source.

11.-12. (canceled)

13. The sterilization indicator reading apparatus of claim 1, further comprising at least one heater block forming the coupling portion of the at least one well, wherein the at least one heating element is thermally coupled to the at least one heater block.

14. The sterilization indicator reading apparatus of claim 13, wherein the processor is further configured to:
control the at least one heating element to maintain a temperature of the at least one heater block at the first preset temperature for the predetermined time duration; and
control the at least one heating element to change the temperature of the at least one heater block to the second preset temperature from the first preset temperature after the predetermined time duration.

15. The sterilization indicator reading apparatus of claim 13, wherein the processor is further configured to:
set a temperature setpoint of the at least one heater block to the first preset temperature for the predetermined time duration;
set the temperature setpoint of the at least one heater block to the second preset temperature after the predetermined time duration; and
control the at least one heating element in order to achieve the temperature setpoint of the at least one heater block.

16. The sterilization indicator reading apparatus of claim 13, wherein the processor is further configured to:
set a temperature setpoint of the at least one heater block to the first preset temperature from the first instance of time;
set the temperature setpoint of the at least one heater block to the second preset temperature upon receiving the second response signal; and
control the at least one heating element in order to achieve the temperature setpoint of the at least one heater block.

17. The sterilization indicator reading apparatus of claim 13, further comprising at least one temperature sensor communicably coupled to the processor and configured to generate one or more signals indicative of a temperature of the at least one heater block, wherein the processor is further configured to control the at least one heating element based on the one or more signals received from the at least one temperature sensor.

18. The sterilization indicator reading apparatus of claim 13, wherein the second preset temperature is greater than the first preset temperature.

19.-21. (canceled)

22. The sterilization indicator reading apparatus of claim 13:
the at least one well comprises a plurality of wells thermally isolated from each other;
the at least one sensor comprises a plurality of sensors, each sensor being configured to sense the at least one parameter associated with the sterilization indicator received at least partially within a corresponding well from the plurality of wells;
the at least one heating element comprises a plurality of heating elements, each heating element being thermally coupled to a corresponding well from the plurality of wells; and
the processor is further configured to independently control each heating element based at least on the first response signal received from a corresponding sensor from the plurality of sensors.

23.-34. (canceled)

35. A sterilization indicator reading apparatus, comprising:
a housing comprising a top portion, and a bottom portion opposite the top portion;
at least one well formed from a portion of the housing, accessible from the top portion, and oriented along a well axis from the top portion to the bottom portion, the at least one well dimensioned to receive at least a portion of a sterilization indicator having spores and a substance fluorescently responsive to a spore viability, wherein the spores are responsive to an environmental condition in a sterilizer, the at least one well comprising a heater block;
at least one heating element thermally coupled to the heater block of the at least one well;
at least one sensor configured to sense at least one parameter associated with the sterilization indicator received at least partially within the at least one well and generate one or more response signals upon sensing the at least one parameter; and
a processor communicably coupled to the at least one heating element and the at least one sensor, wherein the processor is configured to:
control the at least one heating element in order to achieve a temperature setpoint of the heater block;
set the temperature setpoint of the heater block of the at least one well to a first preset temperature from a first instance of time after receiving a first response signal from the at least one sensor;
set the temperature setpoint of the heater block of the at least one well to a second preset temperature:
upon receiving a second response signal from the at least one sensor; or
after a predetermined time duration from the first instance of time; and
set the temperature setpoint of the heater block of the at least one well to a third preset temperature prior to receiving the first response signal;
wherein each of the second preset temperature and the third preset temperature is different from the first preset temperature.

36. The sterilization indicator reading apparatus of claim 35, further comprising at least one temperature sensor communicably coupled to the processor and configured to generate one or more signals indicative of a temperature of the heater block of the at least one well, wherein the processor is further configured to control the at least one heating element based on the one or more signals received from the at least one temperature sensor in order to achieve the temperature setpoint.

37. The sterilization indicator reading apparatus of claim 35, wherein the at least one parameter associated with the sterilization indicator comprises a change in a viability of the spores of the sterilization indicator.

38.-69. (canceled)
